(19) United States
(12) Patent Application Publication
(10) Pub. No.: US 2025/0224414 A1
(43) Pub. Date: Jul. 10, 2025

(54) WIRELESS RETRIEVAL OF BIOLOGICAL SAMPLES

(71) Applicant: THE TRUSTEES OF THE UNIVERSITY OF PENNSYLVANIA, Philadelphia, PA (US)

(72) Inventors: Hyun KOO, Philadelphia, PA (US); Ed STEAGER, Media, PA (US); Alaa BABEER, Philadelphia, PA (US); Minjun OH, Philadelphia, PA (US)

(73) Assignee: THE TRUSTEES OF THE UNIVERSITY OF PENNSYLVANIA, Philadelphia, PA (US)

(21) Appl. No.: 18/935,777

(22) Filed: Nov. 4, 2024

Related U.S. Application Data
(63) Continuation of application No. PCT/US2023/021338, filed on May 8, 2023.
(60) Provisional application No. 63/339,132, filed on May 6, 2022.

(51) Int. Cl. G01N 35/00; A61B 10/02; G01N 1/02

(52) U.S. Cl. CPC G01N 35/0099; A61B 10/02; G01N 1/02; A61B 2010/0216; G01N 2001/028

(57) ABSTRACT

The disclosed subject matter provides systems and methods for retrieving a target sample from a multitude of surfaces including difficult to reach, complex topographies for diagnostic and compositional analyses. The system can include a small-scale robot including a plurality of magnetic nanoparticles (MNPs) and a magnetic control system. The small-scale robot can be magnetically aggregated structure of the MNPs under a magnetic field generated by the magnets or with predetermined shapes, and the small-scale robot can be configured to disrupt and/or retrieve a target sample from a target area through an automated programmable motion and/or positioning.

### CROSS-REFERENCE TO RELATED APPLICATION

This application is a Continuation of International Patent Application No. Application No. PCT/US2023/021338, filed May 8, 2023, which This application claims priority to U.S. Provisional Patent Application No. 63/339,132, which was filed on May 6, 2022, the entire contents of which are incorporated by reference herein.

### STATEMENT REGARDING FEDERALLY-SPONSORED RESEARCH

This invention was made with government support under DE025848 and DE029985 awarded by the National Institutes of Health. The government has certain rights in the invention.

### BACKGROUND

Biofilms can include microbial cells enmeshed in an extracellular matrix and firmly attached to a variety of surfaces. It can be challenging to target certain biofilms formed on surfaces with arbitrary orientations and complex topographical features like crevices. Such complex, biofilm-covered surfaces pervade health care and industry, causing chronic infections and costly contaminations.

Sampling biofilm contents can be important, as the sampled biofilm contents can be analyzed for more effective treatment and selection of precision medicine to increase successful clinical outcomes. However, sampling can be challenging due to the heterogeneous distribution of diverse pathogens that are often located within crevices or grooves.

Accordingly, there is a need for improved techniques and sampling systems to access and remove adhesive biofilms and perform sampling for pathogen and/or pathogenic by-products detection as well as composition analysis.

### SUMMARY

The disclosed subject matter provides techniques for retrieving a target sample. An example system includes a small-scale robot including a plurality of magnetic nanoparticles (MNP) and a magnetic control system including a magnet. In non-limiting embodiments, the small-scale robot can be a magnetically aggregated structure of the MNPs under a magnetic field generated by the magnet. In non-limiting embodiments, the small-scale robot can be configured to disrupt and/or retrieve a target sample from a target area through an automated programmable motion and/or positioning.

In certain embodiments, the magnet can include a permanent magnet, an electromagnet, or a combination thereof. In non-limiting embodiments, the MNPs can include iron oxide nanoparticles (IONPs), nickel, cobalt, iron, or their alloys or oxides.

In certain embodiments, the small-scale robot can be a microscale robot, a milliscale robot, or a centimeter-scale robot. In non-limiting embodiments, the small-scale robot has an extensible and retractable bristle-like configuration. The extensible and retractable bristle-like configuration can be configured to have a topography-adaptive property. The topography-adaptive property can include a stiffness, length, a shape, or a combination thereof.

In certain embodiments, the target area can include grooves, crevices, trenches, space between two or more surfaces, an uneven surface, or combinations thereof. In non-limiting embodiments, the small-scale robot can be configured to reach and conform to the target area. The target area can include any exterior surfaces or enclosed surfaces. In non-limiting embodiments, the IONPs can be configured to be reusable or re-aggregated for an area with different topographies.

The disclosed subject matter also provides methods for retrieving a target sample. An example method includes applying a magnetic field to MNPs, forming a small scale robot by aggregating the MNPs into a predetermined shape, introducing the small scale robot into a target area, and disrupting and/or collecting the target sample from the target area by applying a modified magnetic field that can be configured to induce a motion and positioning of the small scale robot. In non-limiting embodiments, the magnetic field can be configured to induce an automated programmable motion.

In certain embodiments, the method can further include adjusting the mechanical properties of the small-scale robot based on the target area. In non-limiting embodiments, the mechanical properties can include a shape, a length, a stiffness, or combinations thereof.

In certain embodiments, the method can further include analyzing the collected samples for identifying a pathogen, by-products of the pathogen, a composition of the target area, or a combination thereof. The pathogen can include a bacteria, a fungus, a virus, archae, protozoa, algae, and the by-products can include biomolecules and metabolites or combinations thereof.

In certain embodiments, the method can further include reshaping the small-scale robot to reach an area with different topographies. In non-limiting embodiments, the small-scale robot can be configured to reach and conform to the target area. The target area comprises any exterior surfaces or enclosed surfaces.

In certain embodiments, the small-scale robot can be a microscale robot, a milliscale robot, or a centimeter-scale robot. In non-limiting embodiments, the target area can include grooves, crevices, trenches, space between surfaces, an uneven surface, or combinations thereof. In some embodiments, the small-scale robot can have an extended bristle-like configuration.

The disclosed subject matter provides an example system for retrieving a target sample. The example system can include a small-scale robot including a plurality of magnetic nanoparticles (MNP) such as nickel, cobalt, iron or their alloys or oxides. In non-limiting embodiments, the small-scale robot can be a molded structure of the MNPs in a predetermined shape or a 3D printed structure. In some embodiments, the small-scale robot can be configured to disrupt and/or retrieve a target sample from a target area under a magnetic field.

In certain embodiments, the predetermined shape can include a helicoid shape, a vane-like shape, a spherical shape, a spheroid shape, a cylindrical shape, a bullet-like shape, or a spiral-like shape.

In non-limiting embodiments, the small-scale robot can be configured to rotate and translate under the magnetic field. In some embodiments, the small-scale robot is a microscale robot, a milliscale robot, or a centimeter-scale robot.

In certain embodiments, the target area can include smooth surfaces, grooves, crevices, trenches, an uneven surface, or combinations thereof. In non-limiting embodiments, the MNPs can be configured to be reusable or re-aggregated for an area with different topographies.

The method of claim 15, the method further comprising identifying the target sample by detecting components of the sample through an analyzer.

The disclosed subject matter will be further described below, with reference to example embodiments shown in the drawings.

### BRIEF DESCRIPTION OF THE DRAWINGS

FIGS. 1A-1G provide images and graphs showing assembly, control, and functional properties of the surface topography-adaptive robotic superstructure (STARS) in accordance with the disclosed subject matter.

FIGS. 2A-2H provide images and graphs showing the Topography adaptive properties of STARS in accordance with the disclosed subject matter.

FIGS. 3A-3F provide images and graphs showing the physical properties of the assembled bristle-like superstructures in accordance with the disclosed subject matter.

FIGS. 4A-4K provide images and graphs showing biofilm removal, and retrieval of biofilm components using STARS in accordance with the disclosed subject matter.

FIGS. 5A-5E provide images and graphs showing the STARS bristle shape variation and adaptation to human tooth mimic surfaces such as hard to reach areas (e.g. the narrow space between 2 tooth, 5C) in accordance with the disclosed subject matter.

FIGS. 6A-6D provide images and graphs showing automated motion dynamics for biofilm removal and sampling from the natural tooth in accordance with the disclosed subject matter.

FIGS. 7A-7H provide images showing example micro-robotic platforms in accordance with the disclosed subject matter.

FIGS. 8A-8I provide images and graphs showing example functions of the disclosed micro-robotic platforms in accordance with the disclosed subject matter.

FIGS. 9A-9C provide diagrams and graphs showing the assembly, control, and functional properties of robotic nanozyme assemblies and their mode of action in accordance with the disclosed subject matter.

FIGS. 10A-10H provide diagrams and graphs showing the catalytic properties of the nanozyme microrobots in accordance with the disclosed subject matter.

FIG. 11 provides images showing fungal binding in accordance with the disclosed subject matter.

FIGS. 12A-12E provide images and graphs showing programming and automation for precision-guided control of dabbing nanozyme microrobot to a selected target area in accordance with the disclosed subject matter.

FIGS. 13A-13E provide images and graphs showing precision targeting to the infection site and on-site antifungal action on murine mucosa using dabbing nanozyme-microrobots in accordance with the disclosed subject matter.

FIGS. 14A-14E provide images and graphs showing the precision capturing of fungal aggregates using nanozyme-microrobotics techniques in accordance with the disclosed subject matter.

FIG. 15 provides graphs showing time-series images showing dynamic motions of the nanozyme superstructure in accordance with the disclosed subject matter.

FIG. 16 provides images showing catalytic activity in situ in accordance with the disclosed subject matter.

FIG. 17 provides a graph showing the dynamics of catalytic activity in accordance with the disclosed subject matter.

FIGS. 18A-18C provide images and graphs showing the binding of nanozymes on C. albicans biofilms and a confluent human gingival cell layer in accordance with the disclosed subject matter.

FIG. 19 provides time-series images showing the dynamic motions of the dabbing motion.

FIG. 20 provides diagrams showing the coordinate extraction algorithm in accordance with the disclosed subject matter.

FIG. 21 provides a diagram and images showing the quantification of the fungal capturing and removal by computational image analysis in accordance with the disclosed subject matter.

FIG. 22 provides times-series images showing aggregated microswarms that collect the disrupted endodontic biofilm grown on a glass surface in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides systems and methods for retrieving a target sample from a multitude of surfaces for diagnostic and compositional analyses.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The disclosed subject matter provides a diagnostic system for retrieving a target sample. In certain embodiments, the system can include a small-scale robot and a magnetic control system (magnet). The disclosed system can utilize the catalytic and magnetic properties of the small-scale robot for retrieving the target sample (e.g., biofilms) from a target area (e.g., complex surface topographies and difficult-to-reach spaces) such as exterior and interior (endodontic) surfaces of teeth. The disclosed system can be used for diagnostic sampling in medicine, medical device/implant contamination, as well as in environmental/industrial biofouling.

In certain embodiments, the small-scale robot can include iron oxide nanoparticles (IONPs). The IONPs can be versatile materials with catalytic and magnetic properties. The IONPs can perform the peroxidase-like activity that efficiently catalyzes hydrogen peroxide for antimicrobial effects, while the applied magnetic field enables control of the positioning of the IONPs. In certain embodiments, the IONPs can be synthesized via a facile a solvothermal method. For example, iron (III) chloride (e.g., 0.82 g) can be dissolved into ethylene glycol (e.g., 40 ml). Then, sodium acetate (e.g., 3.6 g) can be added to the solution under stirring at room temperature (e.g., for 30 min). Subsequently, the mixture can be transferred to an autoclave and heated (e.g., for 12 h). After the autoclave is cooled to room temperature naturally, the IONP precipitate can be collected, rinsed several times with ethanol, and then dried (e.g., at 60° C. for 3 h). In non-limiting embodiments, the small-scale robot can be a magnetically aggregated structure of the IONPs. For example, under a magnetic field generated by the magnets, the IONPs can be aggregated to form a predetermined shape.

In certain embodiments, the disclosed magnet can generate and control a magnetic field. For example, the magnetic field can be controlled with a device combining programmable electromagnets and servo motors. The magnets can be used to shape and/or reconfigure the aggregated structure of IONPs by a plurality of electromagnets (e.g., coaxially arranged at a distance of 8 mm). Between the two electromagnets, an actuation vessel (e.g., with a dimension of 15×6.7×13 mm³ can be added to accommodate IONPs suspension, and a holder can be constructed to maintain an 8 mm gap between the two electromagnets. Both the electromagnets and servo motors can be mounted around the actuation vessel. In non-limiting embodiments, a ferrite core (e.g., with a diameter of 10 mm) can be situated in the center of the rear electromagnet, and an iron core (e.g., with a diameter of 2 mm) can be positioned in the center of the forward electromagnet. The position of the forward iron core can be moved in varying trajectories using servo motors.

In certain embodiments, the disclosed magnet can be configured to move in various directions and motions. For example, for side-to-side motion, a micro servo motor can be programmed to move (e.g., between 65 and 115 degrees) with an arm (e.g., of 14 mm length). In non-limiting embodiments, a symmetrical arc-shaped movement (e.g., approximately 12 mm) can be implemented with various linear velocities (e.g., from 6 to 48 mm s⁻¹). For circular motion, a continuous rotation servo motor can be used and programmed to allow unrestricted movement at angles using a relatively short arm (e.g., 2 mm length). A continuous circular movement can be generated centered with the axis of electromagnets with a linear velocity (e.g., 12 mm s⁻¹) in clockwise or counterclockwise directions.

For multimodal motions with improved repeatability in position and timing, the system can include a sliding vessel. The body of the device can include a holder for the electromagnets and servos and a sliding vessel. One servo can be used to drive a pinion gear attached to the rack of the sliding vessel for left-to-right motion, and the other servo can be used to drive the forward iron core for circular or arc-shaped motions. The sliding vessel can hold the tooth replicas and natural teeth. In non-limiting embodiments, the vessel can be actuated by a programmable micro servo motor. The motion of the sliding vessel can be coupled with the programmable motion of the electromagnet iron core, which can allow combinations of motions to mimic brushing patterns.

In certain embodiments, the magnetic field can be directed by repositioning of the iron core, and the various positions can be implemented by the movement of the servo controlling the iron core within the forward electromagnet (e.g., side to side and circular by regular and continuous micro servo), during which the magnetic field remained constant. In non-limiting embodiments, the actuation can be performed via the application of a magnetic field and servo programmed to achieve the desired motion pattern. In non-limiting embodiments, the movement of the micro servo arm and the magnetic fields of electromagnets can be controlled using a microcontroller independently using a programmable DC power supply and software for system coding (e.g., Arduino integrated development environment (IDE) software). In non-limiting embodiments, the disclosed magnetic field can be produced by any suitable devices and techniques that can create a magnetic field for sampling or diagnostic purposes.

In certain embodiments, the geometries of the actuation vessel and the body of the magnet system can be modified based on the target area. In non-limiting embodiments, the actuation vessel and the body of the device can be fabricated using a 3D printer (e.g., a low-force stereolithography 3D printer).

In certain embodiments, the small-scale robot is a magnetically aggregated structure of the IONPs. For example, under a magnetic field generated by the magnets, the IONPs can be aggregated to form a predetermined shape. The predetermined shape can include various configurations based on the target area. In non-limiting embodiments, the predetermined shape can include an extended bristle-like configuration. The bristle-like superstructures can be assembled using a magnetic field control system. For example, the dispersed IONPs can be collected by magnetic fields from the forward electromagnet (e.g., for 5 s forming a rounded dome shape), and the strength of the magnetic field used for the standardized biofilm removal experiments can be measured (e.g., to be 90 and 25 mT) at the forward and rear regions, respectively. The small-scale robot can then be designed to orthogonally extend from the forward vessel wall to form elongated bristle-like structures (e.g., for 25 s) by energizing the rear electromagnet. At the beginning of the next cycle, IONPs can be recollected in the forward region. In non-limiting embodiments, a cycle (e.g., a cycle of 30 s) can be repeated up to about 20 times for dental biofilm removal.

In certain embodiments, the appearance and physical/mechanical properties of the small-scale robot can be precisely controlled by the concentration of IONPs (e.g., 0.5 to 1 kg ml⁻¹), the sweep velocity of the servo (e.g., 6 to 48 mm s⁻¹), and the strength of magnetic fields (e.g., 13.3 to 96.4 mT) at the center of the actuation vessel. For example, the appearance and physical/mechanical properties of the small-scale robot can include a shape, a length, a stiffness, or combinations thereof.

In certain embodiments, the disclosed small-scale robot can provide improved reconfigurability and adaptability to various hard-to-reach target areas. The target area can include a square groove, a circular groove, a triangular groove, an uneven surface, crevices, trenches, space between two or more surfaces, or combinations thereof. In non-limiting embodiments, the target area can be an interdental space, the exterior surface of teeth, interior spaces of teeth, or combinations thereof. Aggregated IONPs can conform to these variations through their adaptive nature and can reconfigure to reach distant surfaces in confined spaces as it moves from flat to curved (e.g., through interdental spaces).

In certain embodiments, the shape of the small-scale robot can be formed or reconfigured by the magnet system, depending on the target area. In non-limiting embodiments, the IONPs can be configured to be reusable or re-aggregated for an area with different topographies. In non-limiting embodiments, the small-scale robot can be a microscale robot, a milliscale, or a centimeter scale robot.

In certain embodiments, the small-scale robot assembled from IONPs can be administered to the target area. The small-scale robot can be configured to disrupt and retrieve the target sample. For example, biofilms can be treated with the small-scale robot assembled from IONPs (e.g., 0.5 to 2 mg ml⁻¹ final concentration) solution in the actuation vessel. In non-limiting embodiments, the IONPs can be optimized to enhance the catalytic bioactivity with EPS/biofilm-degrading enzymes (e.g., dextranase/mutanase, 25/5 U) and 1% H₂O₂ (1% v/v). For example, biofilms can be placed in the actuation vessel containing IONP solution for 10 min, followed by the addition of 1% H₂O₂ and subsequent modulation of the magnetic field, which forms the small-scale robot. The velocity, trajectories, and motion frequencies can be controlled by the system. The biofilm removal and retrieval can be performed via the application of an external magnetic field and servo movement preprogrammed to achieve the desired trajectories, velocities, and motion patterns. In non-limiting embodiments, any enzymes, bioactive molecules and/or biologics can be used with the disclosed devices. The types and/or amount of the enzymes, bioactive molecules and/or biologics can be modified depending on the target area.

In certain embodiments, the small-scale robot can disrupt and collect the target sample through its catalytic and actuation activities. The target sample (e.g., biofilm) can be entrenched among the small-scale robots/IONPs. For example, as aggregated IONPs mechanically disrupt biofilms, mixed-species microbial cells (typical of biofilm infections) can bind to the aggregated IONPs. Once removed from the canal, bacterial samples can be analyzed via various chemical, optical, and/or biological analyses (e.g., fluorescent labeling, colorimetric and qPCR assays, DNA/RNA sequencing, omics-based analyses such as proteomics, metabolomics). For example, the genomic DNA can be extracted and purified from retrieved samples, and qPCR can be performed with specific primers to detect each bacterial species.

In certain embodiments, the disclosed subject matter can provide real-time analysis. The samples retrieved can be analyzed in real-time via facile colorimetric, electrochemical or fluorescence assays that can be detected using readily available sensors integrated into a device. For example, the pathogens captured by the robots can react with specific fluorophores or chromogenic substrates that can be detected via small detectors embedded in a device. This can include combinations of light-emitting diodes (LEDs) and photodetectors to assay optical absorbance, emittance and reflectance. Conversely, IONPs can be functionalized with coatings (e.g., antibodies, polymers, and proteins) that bind to a specific pathogen, allowing in situ detection via colorimetric analysis. Because the outcome provides both qualitative (e.g., yes or no) and quantitative (e.g., levels/amounts) data, simple algorithms can be programmed into apps or integrated into the disclosed device. In non-limiting embodiments, the disclosed subject matter provides improved design flexibility. For example, a small tray- or mouthguard-like device containing colorimetric or fluorescence sensors, which can detect the pathogens or other biofilm components retrieved by the small robots brushing the teeth, can be included.

In certain embodiments, the small-scale robot can be assembled through a 3D mold. For example, the small-scale robot can be a molded structure of the IONPs. The molded robotic system with a predetermined shape can be developed using 3D-printed negative molds using the IONPs as building blocks. In non-limiting embodiments, the predetermined shape can be a helicoid shape. The 3D molded helicoid robots (e.g., two helices wrapped around a central axis) can be formed by embedding IONPs within a thermo-reversible granulated agar gel in a final soft robot composition (e.g., contains 25% glycerol, 3% (w/v) agar gel within which 10% IONPs are embedded). The molding technique can be employed with other hydrogels or polymers. The IONPs-embedded gel can be used to fill the negative molds to create 3D molded soft helicoids (e.g., with a size of ˜700 μm by 1.5 mm). The size can be modified depending on the target area. In non-limiting embodiments, the molded small-scale robot can be 3D printed directly. For example, 3D printers can be used for printing a mixture of photoreactive polymer with nanoparticles, which can provide improved design flexibility.

In certain embodiments, when directed by the magnetic fields, the 3D molded helicoids can be configured to propel and/or rotate. For example, when a magnetic torque is applied, a 3D molded helicoid can rotate around its central axis. This can cause forward locomotion due to the chiral geometry and corkscrew-like action of the helicoid. The helicoid robot can be adapted to access and sample biofilms from other similarly challenging spaces and geometries, such as in the interior of catheters and water lines/pipes.

In certain embodiments, the 3D molded robots can also be used for biofilm diagnostics. For example, for endodontic applications, biofilms can be detected in apical spaces by retrieving samples via physical disruption and adhesion to the 3D molded robot exterior. The collected sample can be analyzed by various chemical and/or biological analyses (e.g., colorimetric, electrochemical, fluorescent labeling and qPCR). The bacteria disrupted by the front end can adhere to the back end of the molded robot via interaction with a dextran coating or other adhesive surface. As with aggregated IONPs, samples are analyzed using fluorescence probes for each species and qPCR with species-specific primers to detect the presence of different bacterial species.

In certain embodiments, the disclosed subject matter provides a method of retrieving a target sample using the disclosed system. The method can include applying a magnetic field to iron oxide nanoparticles (IONPs), forming a small scale robot by aggregating the IONPs into a predetermined shape, introducing the small scale robot into a target area, and disrupting and/or collecting the target sample from the target area by applying a modified magnetic field.

In non-limiting embodiments, the method can further include adjusting the mechanical properties of the small-scale robot based on the target area. In non-limiting embodiments, the mechanical properties comprise a shape, a length, a stiffness, or combinations thereof. The mechanical properties can be modified by controlling the concentration of IONPs, the sweep velocity of the servo, and the strength of magnetic fields.

In non-limiting embodiments, the target area includes any hard-to-reach area with challenging spaces and geometries. For example, the target area can be an interdental space, the exterior surface of teeth, the interior spaces of teeth, the interior of catheters, interior spaces of water lines/pipes, containers/tanks, medical implants, dentures, medical devices, household surfaces, glass surfaces, or combinations thereof.

In certain embodiments, the method can further include analyzing the collected samples for diagnosing a pathogen and/or its by-products. The pathogen can include bacteria, fungi, viruses, archae, protozoa, algae, microial, and the by-products can include biomolecules and metabolites or combinations thereof. The sample can be analyzed via various chemical or biological analyses. For example, the analyses can include a qPCR analysis, a colorimetric assay, a fluorescent labeling analysis, omics-based analysis or a combination thereof.

In certain embodiments, the method can further include reshaping the small-scale robot to reach an area with different topographies. The disclosed small-scale robot can be reusable and configured to be re-assembled through the disclosed magnet system. For example, after collecting the target sample, the small-scale robot can be disassembled and washed. The disassembled IONPs can then be re-assembled for the next target area with different topographies.

The disclosed subject matter provides micro to milliscale robots that can be precisely guided to access difficult-to-reach spaces and complex surface topographies, including angled grooves or narrow crevices. The disclosed system can be used for (1) diagnostics via retrieval and sampling of biofilms associated with infectious diseases, (2) assessment of biofouling of dental/medical devices or implants, (3) compositional analysis of the samples, including microbial composition, by-products and biomolecules, including sugars, proteins, enzymes, polysaccharides, metabolites and (4) analysis of materials for non-medical applications (e.g., industrial application). The disclosed system using the magnetic robotic actuation can allow tether-free controlled motion and enables a wide variety of motility and locomotion strategies. In non-limiting embodiments, the operation of the disclosed robots can be controlled by an automated system.

The disclosed subject matter provides a nanozyme-based microrobotics platform that directs localized catalysis to an infection site with microscale precision to achieve targeted fungal binding. For example, the disclosed subject matter provides a set of structured assemblies composed of iron oxide nanozymes that deliver and localize antifungal activity through dynamic shape transformation and catalysis activation using electromagnetic field frequency modulation and fine-scale spatiotemporal control. The disclosed nanozyme assemblies can display controllable catalysis that varies depending on the location, motion, and velocity, providing tunable and precision-guided reactive oxygen species (ROS) generation.

In certain embodiments, the disclosed nanozyme assemblies can bind avidly to a target (e.g., fungal cells), coating the microbial surface to enable the localized in-situ targeting of biofilms. For example, the structured nanozyme assemblies can be directed to C. albicans-infected sites by programmable robotics and control to perform precisely guided fungal targeting.

The disclosed subject matter also provides microrobots for tetherless biofilm treatment and retrieval using iron oxide nanoparticles (IONPs) with dual catalytic-magnetic functionality as building blocks. For example, an example system can be formed from IONPs that assemble into aggregated microswarms under magnetic fields that can be controlled to disrupt and retrieve biofilm samples for microbial analysis. In non-limiting embodiments, an example platform can be composed of 3-dimensional (3D) micromolded opacifier-infused soft helicoids with embedded catalytic-magnetic IONPs that can be visualized via existing radiographic imaging techniques and controlled magnetically inside the root canal, uninterrupted by the soft and hard tissues surrounding the teeth in an ex vivo model. These microrobots placed inside the root canal can remove biofilms and be efficiently guided with microscale precision.

The disclosed subject matter can be used for various industrial applications. For example, the disclosed robot can be used for any hard-to-reach area with challenging spaces and geometries to retrieve the target sample in the beverage, water, and pipe industries. For example, the target area can be interior spaces of water lines/pipes, household surfaces (swimming pools), glass surfaces, or combinations thereof. For example, industrial applications include food and beverage processing systems and water treatment systems.

In non-limiting embodiments, the amount and/or the size of the disclosed robot can be adjusted for industrial applications. For example, the size of the robot can range from about 1 nm to about 5 m depending on the target sample and/or the industrial application. For example, processing and treatment facilities for food, water, energy and chemical production. The amount of the robot can range from about 1 mg to about 1 kg depending on the target sample and/or the industrial application. For example, sampling of microbes from industrial water treatment or beverage production facilities.

### EXAMPLES

### Example 1: Surface Topography-Adaptive Robotic Superstructures for Pathogen Detection and Biofilm Removal on Human Teeth

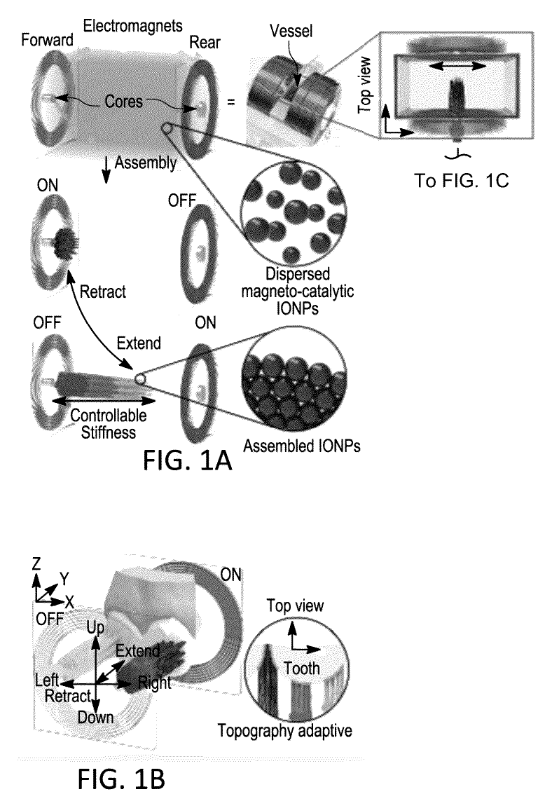

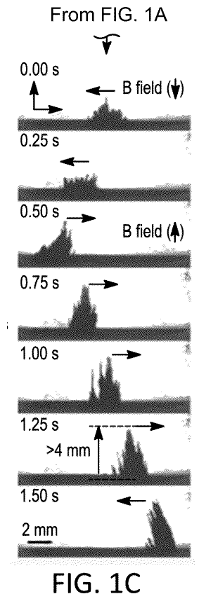

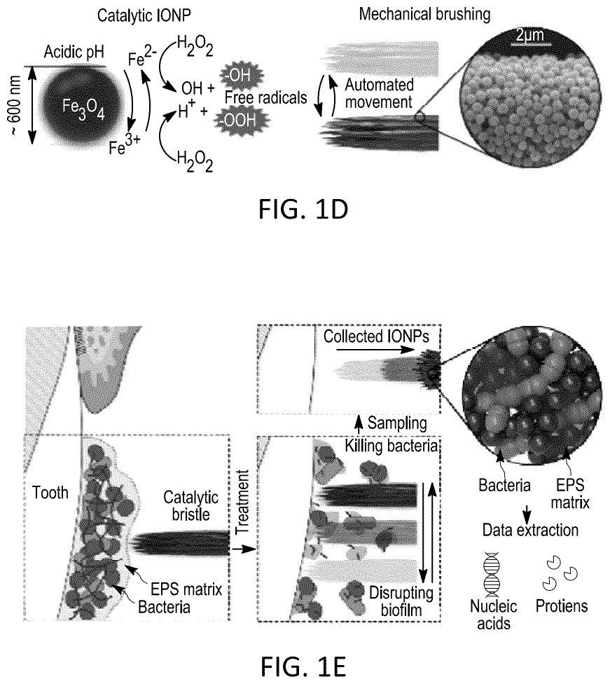

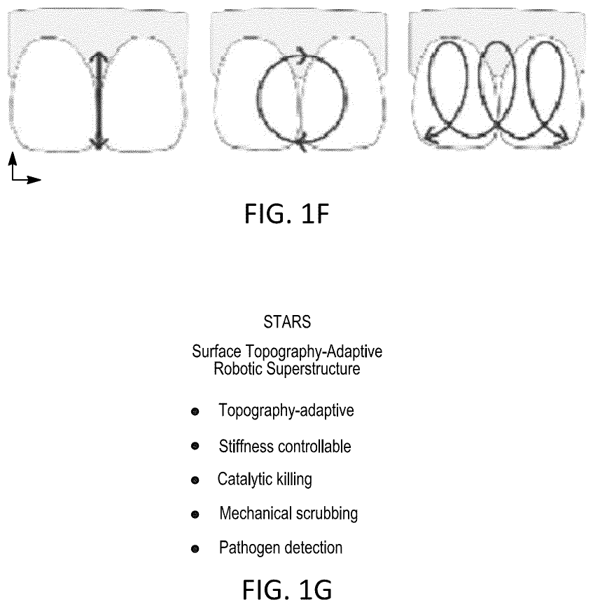

Development of reconfigurable STARS: an automated electromagnetic platform was designed to treat biofilms on vertically oriented surfaces, which requires self-supporting functional elements that withstand gravitational forces. As illustrated in FIG. 1, a field-directed technique was developed to dynamically assemble and actuate magnetic bristles from IONPs in solution. These bristle-shaped superstructures can extend horizontally from a vertical base to interact with an opposing vertical surface (FIGS. 1A and 1B).

The components of the control system are arranged around a customized vessel that holds an aqueous IONP solution (FIG. 1A). FIG. 1A shows that IONPs can be dispersed in solution between two electromagnets. IONPs were collected on the forward wall of the vessel, then extended into a bristle-like superstructure with controllable stiffness. FIG. 1B shows that the forward electromagnet core can guide the bristles across the target surface with the topography-adaptive property. FIG. 1C shows the top-view of IONPs (1 mg ml⁻¹) that were initially collected in a low aspect ratio mound and then extended into a high aspect ratio bristle-like formation as they were swept laterally. FIG. 1D shows that IONPs are multifunctional with peroxidase-like activity, generating free radicals at the site of mechanical cleaning and providing both antimicrobial treatment and physical biofilm removal. FIG. 1E shows that bristle motion can be controlled to disrupt biofilms through mechanochemical action and retrieve biofilm contents (e.g., microbes, extracellular polysaccharides, biomolecules) for diagnostic sampling. FIG. 1F shows that programmable motions of the disclosed subject matter enable targeted treatment and optimized cleaning via automated routines. FIG. 1G shows multifunctional and multitasking capabilities integrated into surface topography-adaptive robotic superstructure (STARS).

A set of programmable electromagnets positioned on either side of the vessel controls the formation and multimodal action of the magnetic bristles. The magnetic field generated from both electromagnets is coordinated via a programmable microcontroller. Furthermore, the position of the forward iron core can also be controlled while the rear ferrite core is fixed. This design enables flexible positioning and control of bristle-like superstructures that form on a vessel wall and can span the vessel width while moving in multiple directions as directed by the local field. This allows automation of both motions and positioning of the robots.

For example, in one modality, the magnetic field is oriented to drive the superstructure assembly with its base on the vessel wall near the mobile forward electromagnet core in the xz-plane (FIG. 1A), extending in the y-direction (orthogonally) toward the rear electromagnet. By cyclically changing the position of the forward magnetic core, the superstructure position can be controlled. The resulting bristles can enable biofilm removal from a vertically oriented surface placed in the vessel, such as the human tooth (FIG. 1B). When viewed from above, the bristles extend during the first 1-2 seconds of the cycle oriented toward the rear electromagnet and translate across the vertical vessel wall (FIG. 1C); the superstructure self-supports as the length increases during the lateral motion. Furthermore, IONPs, which are catalytic with a peroxidase-like activity that can activate hydrogen peroxide (H₂O₂), were used to produce free radicals on-site for antimicrobial activity (FIG. 1D).

Such magneto-catalytic properties combined with magnetic field modulation allow automated, reconfigurable bristles to be formed with multiple programmable functionalities, including extension and retraction, topographical adaptability, and tunable stiffness gradients. These surface topography-adaptive robotic superstructures (STARS) chemically treat and mechanically remove biofilms (FIG. 1D), and can penetrate biofilms to retrieve samples for diagnostic analysis (FIG. 1E). Additional automation of STARS bristles placement enables varying motion dynamics with unique spatial (multi-axis) control that can be evaluated for the most efficient biofilm removal and localized diagnostic sampling on the tooth surface (FIG. 1F).

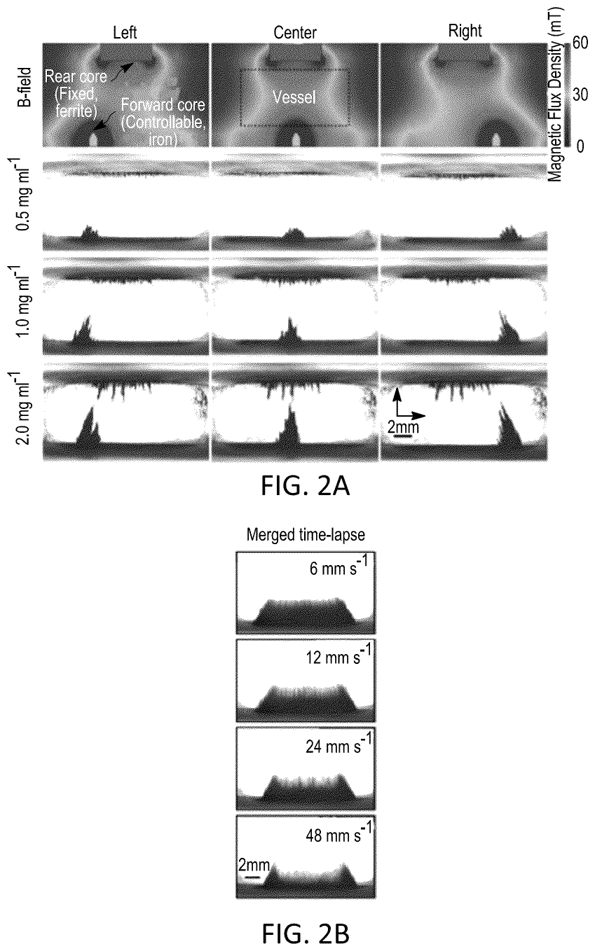

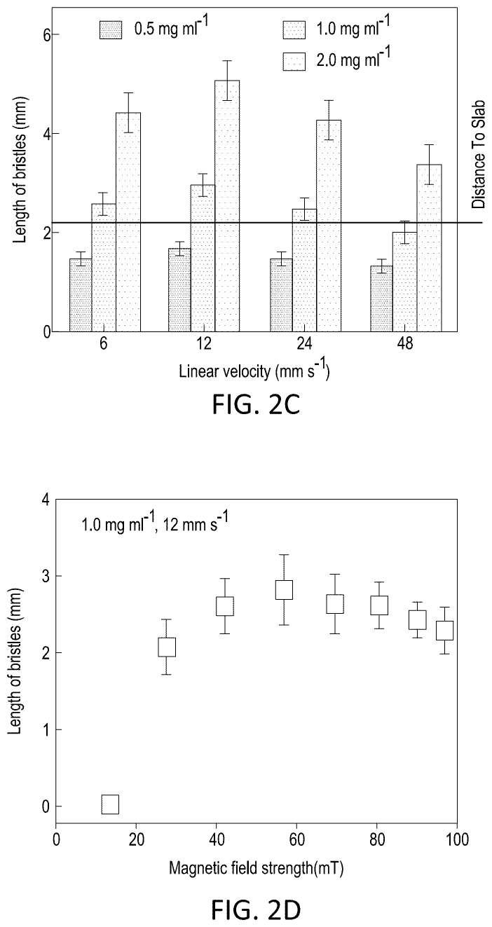

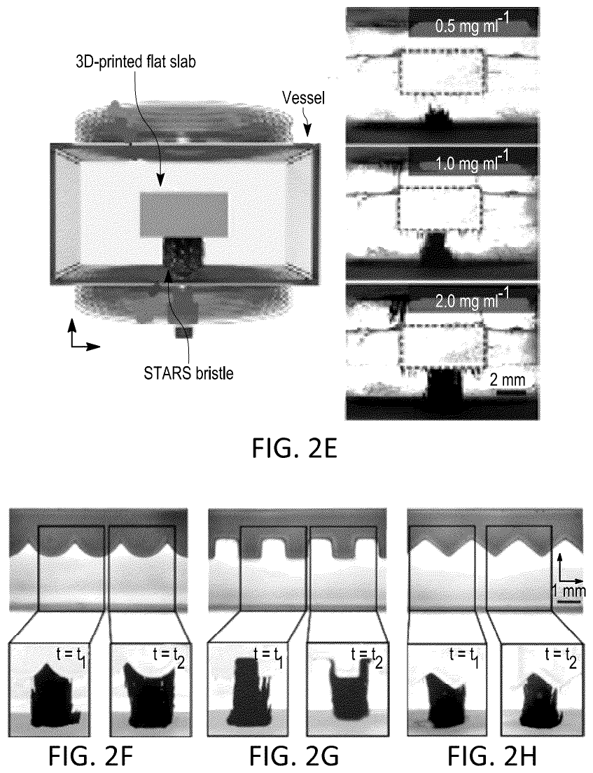

Physical characterization of STARS: The magnetic field generated by the electromagnets enables control over bristle formation, position, and properties. A 3D finite element model was developed to better understand the balance of magnetic fields and forces that control these properties and compare the prediction to the experimental outcome. As shown in FIG. 2A, STARS bristles align along the direction of the magnetic field, extending from the position of the positionable forward electromagnet core toward the fixed rear electromagnet. The values for magnetic flux density in the center of the vessel, where the vertical biofilm-covered surface can be situated, vary from 35-70 mT, depending on the instantaneous position of the forward core (FIG. 2A). Electromagnets are cycled on and off during the cleaning cycle enabling three important aspects of control. Firstly, IONPs can be flexibly reconfigured into self-supporting bristles on the vertical surface. Secondly, as they form, bristles can be pulled into the biofilm, enhancing mechanical interaction. Thirdly, by toggling the field on and off, the bristles can be released and re-assembled at will, allowing structure formation, release, and reconfiguration at tunable locations.

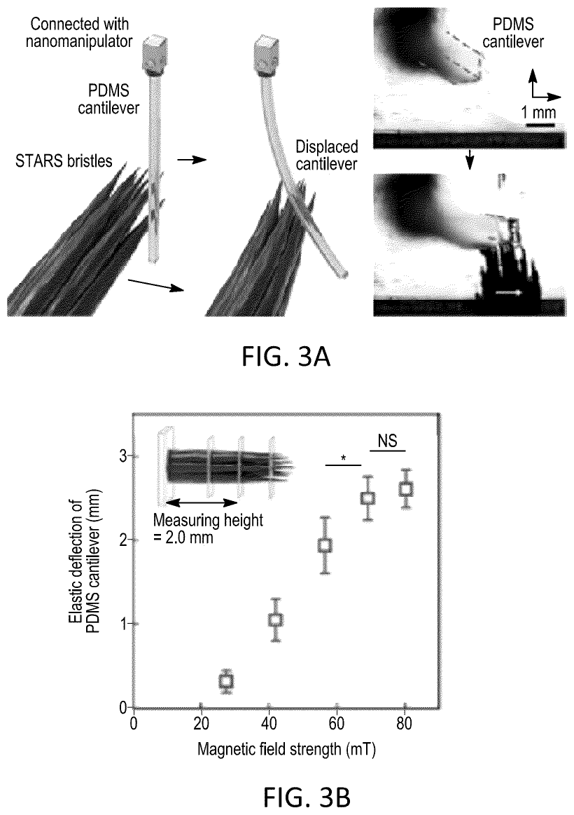

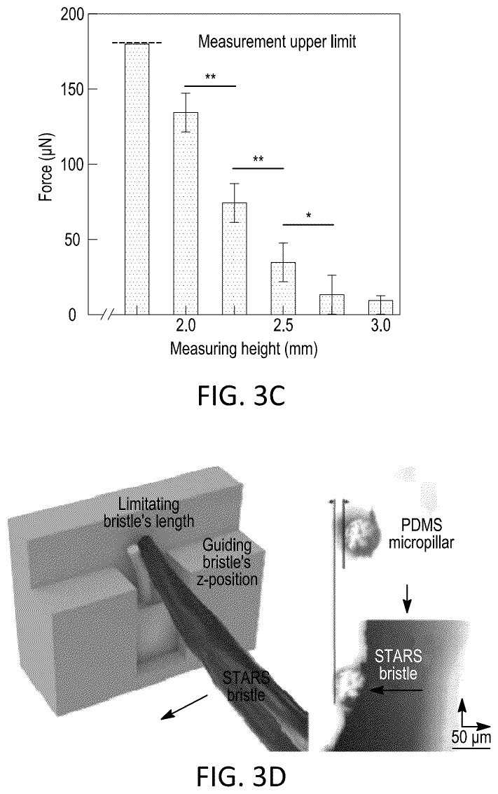

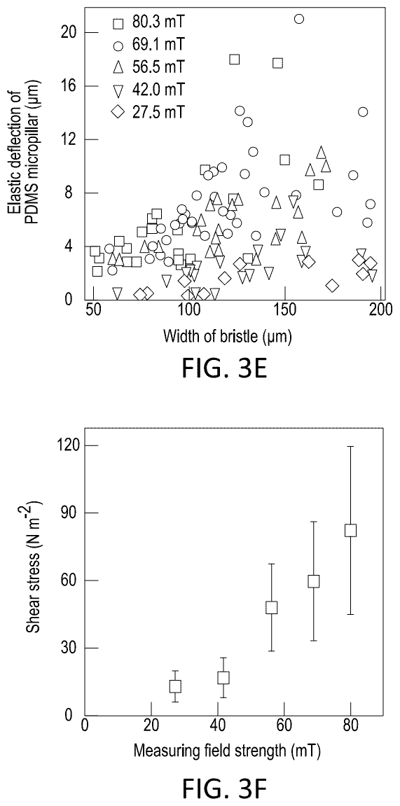

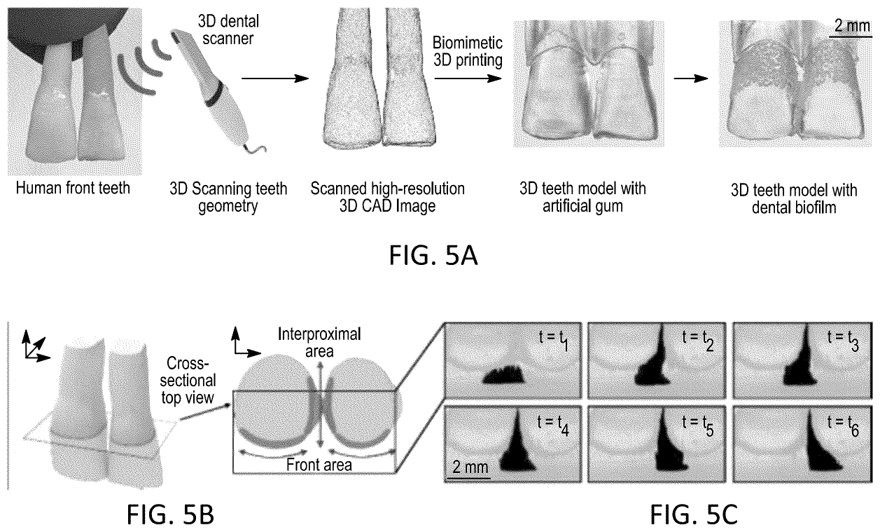

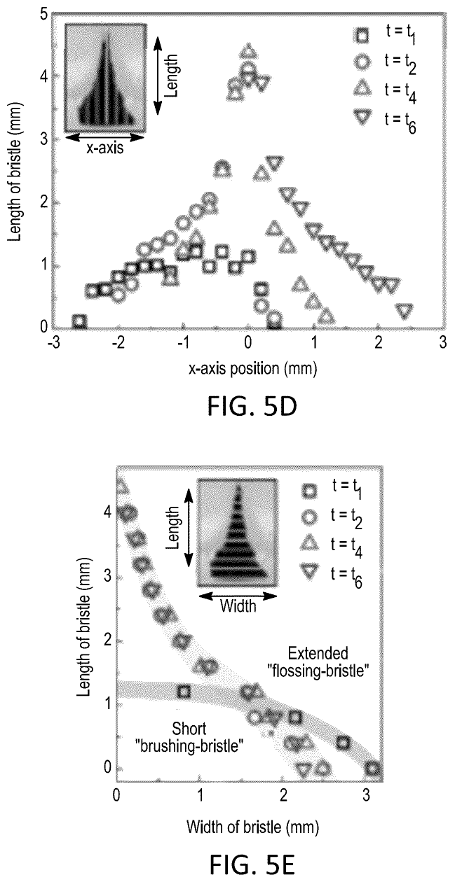

The magnetic fields afford dynamic bristle control. Bristle alignment is controlled using fields from both electromagnets, and the bristle position is tuned using magnetic forces generated by the mobile forward electromagnet core. As these forces sweep laterally across the vessel wall, the bristles follow, allowing brushing motions (FIG. 2A). IONPs dispersed in an aqueous solution were used at a concentration of 1 mg ml⁻¹. IONPs are dispensed into the 1.3 ml vessel and observed via time-lapse microscopy. Upon energizing the forward magnet, the assembly of suspended bristles on the forward wall of the vessel was observed (FIG. 2A). IONPs collect into a cohesive superstructure toward the end of the forward electromagnet core (B=90 mT), resulting in a rounded dome of IONPs approximately 300-800 μm in height, shown at 0 s in FIG. 1C. After initial assembly, the rear electromagnet is energized while the forward electromagnet is de-energized. Under the combined magnetization in both cores, the rounded dome extends into bristle-like structures extending orthogonally from the forward vessel wall. FIG. 2A shows finite element modeling (top row) demonstrating the deformation of the magnetic B-field by changing the positioning of an electromagnet core (rows 2-4). Bristle position forms at the site of the high B field, and bristle length varies with the initial concentration of IONPs. FIG. 2B provides time-lapse composite images (1 mg ml⁻¹ IONPs) showing the height of the bristles across different positions over time. FIG. 3C shows that greater bristles height can be achieved by increasing IONP concentration and reducing the velocity of lateral actuation. FIG. 3D shows that bristle length can vary with magnetic field strength. FIG. 5E shows an example 3D-printed platform that was developed to mimic the vertical positioning of the tooth (top row). Suspensions containing 1-2 mg ml⁻¹ IONP are sufficient to reach the target surface. FIGS. 2F-2H show that STARS bristles can reconfigure and self-conform to different surface topographies (e.g., circular, square, triangular).

The core of the forward electromagnet is then actuated to move bristles in a lateral motion repeatedly across a vertically oriented surface in a sweeping motion with linear velocities between 6 and 48 mm s⁻¹. Since the forward core is small in diameter (3 mm), the magnetic forces are focused in a small region and bristles closely track its movement. The rear ferrite core is larger in diameter (10 mm) and generates weaker forces that serve primarily to define the direction of bristles' extension. As the forward core translates, the bristle closely follows the highest gradient of the magnetic field, as shown in the finite element model (FIG. 2A). To collect IONPs at the end of a cleaning sequence, both coils are de-energized to allow the bristles to dismantle and settle to the bottom of the vessel, which occurs within 1-2 seconds. After the bristles settle, the forward coil is re-energized, and the cycle is repeated. To summarize, bristle assembly and motion dynamics involve three sequential and interdependent processes. In the initial process, IONPs are collected by energizing the forward electromagnet. Thereafter, the rear electromagnet is energized, which guides bristles formation and extension toward the position of the vertical biofilm-contaminated surface. Finally, the lateral motion creates changes in bristle shape and length guided by the magnetic field.

Bristle length depends on the IONP suspension concentration and can range from 1 mm in length at 0.5 mg ml⁻¹ to as long as 7 mm at 2 mg ml⁻¹. The bristle shape can be influenced by the movement of the electromagnet core, extending during the first few seconds (FIG. 1C). The effective length of the bristles can be visualized by creating a composite of accumulating sequential images of the sweeping process (FIG. 2B). Bristle length can also depend on their lateral velocities; at higher velocities, fluid drag forces disrupt the cohesion among IONPs at the distal end of bristles resulting in shorter lengths (FIG. 2C). Bristle length can also show non-monotonic dependence on magnetic field strength, first extending with magnetic field strength but then retracting at higher field strengths as IONPs pack tightly (FIG. 2D). A bristle length of 2.2 mm is sufficient to reach the planar vertical surfaces placed in the vessel between the two electromagnets that are used for characterizations disclosed herein, where IONP concentrations at or above 1 mg ml⁻¹ increase the surface coverage (FIG. 2E).

Given the extensibility of the bristles, the bristle interaction with topographically complex surfaces was assessed. Using lithographical methods, surfaces with repeated circular, square, and triangular patterns were generated (FIG. 2, F to H), and the bristles can readily reach and adapt to enter the surface recesses, including corners in the square patterns and cusp-like features at the intersection between the two circular shapes. This novel shape-adjusting, topography-adaptive functionality is a form of physical intelligence that can enhance the ability to remove biofilms formed in difficult-to-reach surfaces.

Bristle mechanical properties can be characterized using two complementary methods, both of which are based on force estimates derived from using Euler's beam deflection approximation:

$$F = \frac{{δ}_{b} {L}^{3}}{3 EI} \qquad ( 1 )$$

Where δ_{b} is deflection, L is beam length, E is Young's modulus, and I is the area moment of inertia. First, the cohesion force of STARS bristles was measured using a PDMS microcantilever to determine the physical strength of the bristle itself (FIG. 3A). The deflection of the cantilever tip by the lateral sweeping motion of the bristles is recorded. Using known values of the length, Young's modulus and moment of inertia of the microcantilever, the force exerted by the bristles on the microcantilever, and therefore the reaction force exerted by the PDMS microcantilever on the bristles is determined. The ability of the bristles to withstand this reaction force can be used to characterize STARS bristle mechanics. The force was increased until the bristles broke, allowing the cohesive strength to be determined. The maximum applied force was measured at a fixed position while varying field strength. There is a linear relationship between the lateral force applied by the bristles and magnetic field strength (FIG. 3B). This demonstrates the feasibility of dynamically tuning bristle stiffness during operation. By placing the microcantilever at various positions along the long axis of the magnetic bristles at a constant field strength of 69 mT, cohesion forces were estimated ranging from 8.2 μN at the distal end to 180.5 μN near the base of the bristles (1.75 mm measuring height, FIG. 3C). Furthermore, using the value for applied shear force, the applied shear at the surface (τ=F/A, A is the contact area) was measured by estimating the applied cross-sectional area of the bristles at the interface. Using the force measurements obtained using the PDMS microcantilevers, shear stresses ranging from 10.4 to 229.8 N m⁻² were measured.

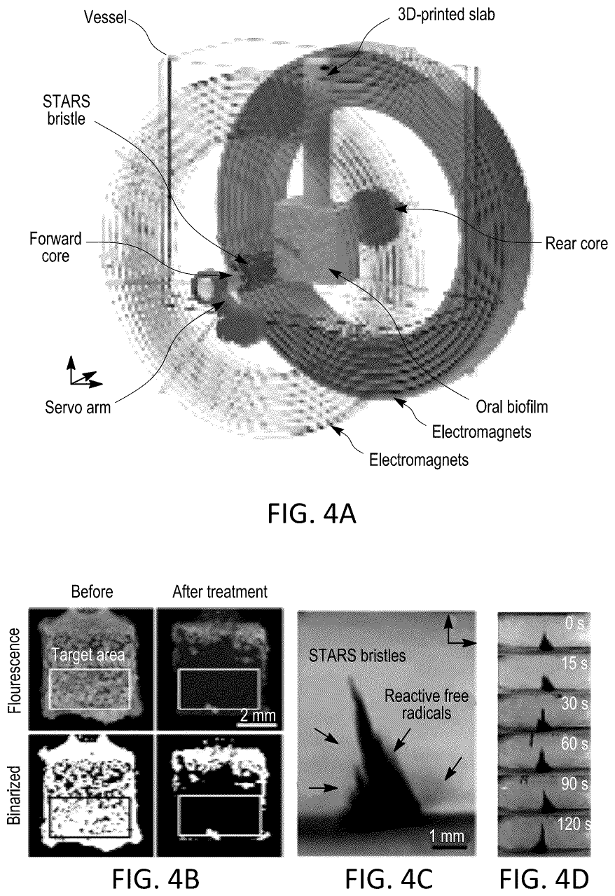

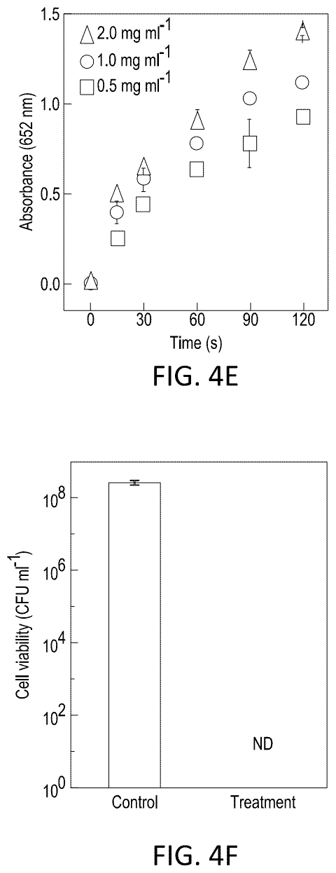

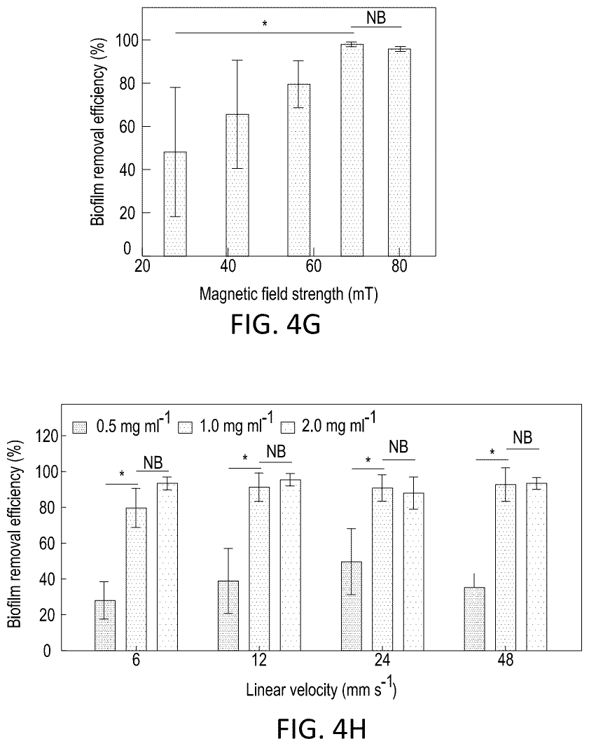

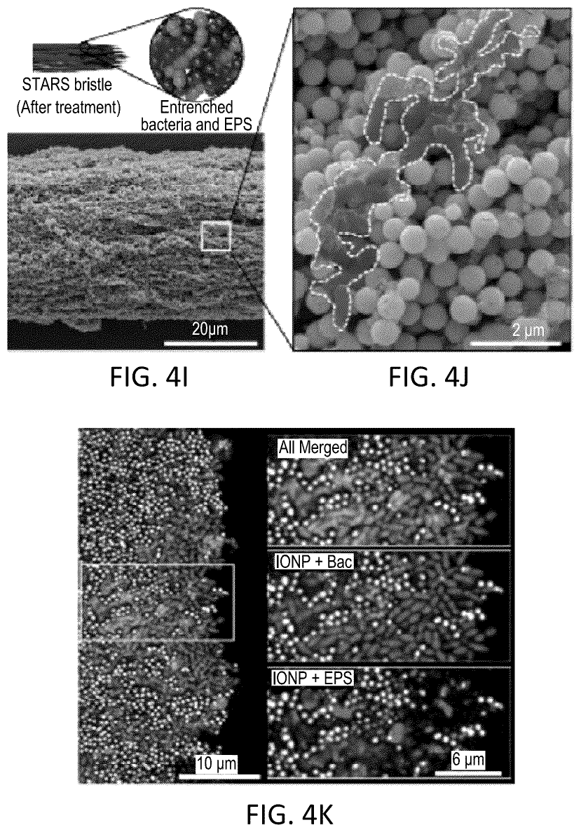

FIG. 3A shows magnetic bristle (1 mg ml⁻¹, 12 mm s⁻¹) strength evaluated by measuring the deflection of PDMS microcantilevers (1.0×1.0×22 mm³). FIG. 3B shows that lateral force increases linearly with magnetic field strength. Data are mean±s.d., *P represents <0.0001; NS means not significant; and P=0.6017 (n=12). FIG. 4C shows the estimated lateral force of STARS bristles depending on the measuring height. Data are mean±s.d., *P=0.0025, **P<0.0001 (n=10). FIG. 3D shows forces applied to the targeted surface that was evaluated using a PDMS micropillar (50 μm of diameter and 275 μm of height). FIG. 3E shows Elastic deflection, and FIG. 3F shows shear stress applied at the surface by STARS bristles (1 mg ml⁻¹, 12 mm s⁻¹). Data are mean±s.d.; one-way analysis of variance (ANOVA) followed by post-hoc Tukey's test.

At the interface of the STARS bristles with the treatment surface, the dominant stresses are the lateral shear stress applied by the bristles, which is opposed by a combination of fluid drag and adhesion between the biofilm and the substrate. Unlike typical monolithic bristles, the IONPs near the bristles end continuously reconfigure and change the bristle structure under high shear near the vertical surface. To verify that these dynamic structures can produce significant stresses, a micropillar composed of PDMS with 50 μm diameter and 240 μm height (aspect ratio=4.8) was used. This single micropillar interacts with the small-scale features at the ends of the bristles allowing characterization of the local force generated by these structures (FIG. 3D).

The micropillar is vertically placed at the position of the target surface and imaged during bristle contact to measure the deflection. Based on its deflection, the shear stress applied at the 2.2 mm distance to be as high as 83.3 N m⁻² was measured depending on the magnetic field strength (FIG. 3, E and F). The shear stresses measured by both the cantilever and pillar measurement systems are comparable in magnitude and demonstrate that the shear stresses produced at the vertical target surface or tooth surface are significantly greater than the minimum values necessary for biofilm removal, even in narrow, difficult-to-access spaces. In addition, the shear stress can be adjusted to remove biofilms at different distances from the surface by adjusting magnetic field strength.

STARS bristle movement is opposed by viscous drag, which increases with actuation speed. This contact force was measured as a drag on a cylindrical body at constant velocity. The Reynolds number,

$$R e = \frac{ρ u L}{μ} ,$$

is on the order of 10 during movement, well within the laminar flow regime, where ρ and μ are the density and dynamic viscosity of the fluid, u is the flow speed, and L is the characteristic linear dimension. The viscous drag force on the translating bristle can be estimated as

$${f}_{D} = \frac{1}{2} {C}_{D} ρ {u}_{0}^{2} A ,$$

where the drag coefficient C_{D} is ˜5, u₀ is the velocity of the bristle, and A is the area of the bristles facing the fluid. This yields an estimated maximum drag force on the bristle of 0.8 μN at 12 mm s⁻¹, which is an order of magnitude below the applied shear force at the vertical target surface/bristle interface.

These complementary analyses reveal four essential properties of STARS bristles. (i) The support structure varies along the length of the superstructures, with a highly cohesive base that supports the extended bristles with decreasing yet strong cohesion along the length of the structure. (ii) The cohesion and associated capability of the bristle to mechanically remove strongly adhesive biofilms is retained at the distal end, where stresses exceed the values necessary for biofilm removal. (iii) Even as the STARS bristles reconfigure and adapt to fine-scale surface topography, applied shear stress remains sufficient to remove biofilm, as evidenced by shear stress measurements of the smallest bristles. (iv) This shear stress can be dynamically adjusted by modulating the magnitude of the magnetic field, enabling fine-tuning of the maximum shear for controlled biofilm removal. Hence, the ‘stiffness gradient’ provides strong cohesion at the base of the bristle to withstand gravity and extend horizontally, while flexibility at the tip provides enough shear force to dislodge biofilms while continuously adapting to the topography.

Antibiofilm functionality of STARS: The process of STARS bristle assembly and the physical properties establish the fundamentals for targeted magneto-catalytic action for biofilm treatment and sampling. An oral pathogen (Streptococcus mutans) was used to form one of the stickiest biofilms with matrix-enmeshed bacteria, which is remarkably difficult to remove. Next, the removal of biofilms on vertically oriented surfaces was assessed. To mimic the anatomical positioning of teeth in the upper arch, slabs are 3D-printed and placed in a vertical position similar to the upper incisors (FIG. 4A). Biofilms of S. mutans are formed on the slabs (see Materials and Methods for details). The slab material's surface characteristics have been verified to have similar biofilm adhesion properties as human enamel, i.e., requiring 0.184 N m⁻² for biofilm removal.

FIG. 4A shows a schematic of the experimental platform for measuring the efficacy of biofilm cleaning: the physical dimension (width×depth×height) of the slab is 4.0×2.2×4.0 mm³. FIG. 4B shows the biofilm cleaning efficacy that was evaluated across the targeted area (4.0×2.0 mm²) using fluorescent labeling and binarized image analysis. FIGS. 4C-4E show catalytic activity in situ. TMB assays demonstrate the generation of reactive oxygen species on-site from H₂O₂ by the catalytically active (peroxidase-like) STARS bristles (1 mg ml⁻¹, 12 mm s⁻¹). FIG. 4F shows cell viability counts that illustrate disruption of targeted biofilms via catalytic activation of H₂O₂. ND means not detectable. FIG. 4G shows that biofilm removal efficacy can increase as the magnetic field and bristle stiffness increase. Data are mean±s.d., *P=0.0249; NS means not significant P>0.9999 (n=3). FIG. 4H shows that biofilm removal efficacy can increase with IONP concentration and can be correlated with scrubbing velocity. FIG. 4I shows that biofilm components (microbial cells and extracellular material) can become entrenched among the assembled STARS bristles. FIGS. 4J and 4K show SEM and confocal images demonstrating the entrenchment and retrieval of biofilm components, i.e., bacteria (Bac) and extracellular polysaccharides (EPS). Data are mean±s.d., *P<0.0001; NS means not significant P>0.05 (n=4); one-way ANOVA followed by post-hoc Tukey's test.

To characterize biofilm scrubbing on the surface, an automated, standardized process was developed to ensure consistency and repeatability. During forward surface biofilm removal, the geometry was fixed, and the magnetic field strength was held constant at 69 mT for focusing on two parameters, specifically, IONP concentration and scrubbing velocity. The distance between the vessel wall and the biofilm specimen during all experiments was fixed at 2.2 mm. IONP suspensions at concentrations ranging from 0.5 to 2.0 mg ml⁻¹ were added to the vessel. A uniform 10 mm lateral sweeping motion is used to quantify biofilm removal at all IONP concentrations and sweeping velocities. This focused motion was intended to provide a quantifiable metric for efficacy rather than to remove the biofilm from the entire surface. As such, biofilm removal was measured in terms of pre- and post-treatment based on binarized before/after images using a threshold value (FIG. 4B).

Given that IONPs display peroxidase-like activity, STARS bristles can provide an on-site source of free radicals for catalytic reaction-generated antimicrobial effects. A colorimetric assay using 3,3′,5,5′-tetramethylbenzidine (TMB) was used to demonstrate the generation of reactive oxygen species from H₂O₂ by the catalytically active STARS bristles. The hydroxyl radicals produced from H₂O₂ oxidize colorless TMB to blue-colored reaction products, which can be visualized and assayed by measuring the absorbance at 652 nm. The catalytic activity of the STARS bristles is determined in the same conditions as the biofilm removal assay. The assay indicates the rapid generation of reactive oxygen species during the first two minutes of cleaning (FIG. 4, C to E). The catalytic activity can be readily visualized in the close-up image showing a free radical reaction with TMB (in blue) immediately surrounding the bristles in real-time, which accumulated over time (time-lapse panel). The free radicals generated from catalytic activity can chemically kill bacteria embedded in the biofilms. The cell viability (viable counts) of the removed biofilms post-treatment was determined. The data show no detectable viable cells following treatment with the STARS bristles in the presence of H₂O₂(FIG. 4F); however, the removed biofilm from the control group harbored more than 10⁸ CFU ml⁻¹ of viable cells after bristle treatment without H₂O₂, demonstrating the efficacy of the magneto-catalytic bristles for biofilm removal.

Biofilm removal efficacy is first evaluated as a function of magnetic field strength (FIG. 4G). Biofilm removal is quantified by capturing images using fluorescent labeling, binarizing the targeted region, and calculating the portion of biofilm removed from the targeted region (FIG. 4B). Removal efficacy increases as bristle stiffness increases until the field strength becomes great enough to compact and slightly retract the bristle (FIG. 2D). Optimized parameters for further biofilm removal experiments were based on this result. As IONP concentration increases, the availability of IONPs in suspension for bristle formation increases, which corresponds to greater length after bristle assembly. At the lowest concentration (0.5 mg ml⁻¹), bristles formed with lengths between 1 and 2 mm and were shown to have limited efficacy at scrubbing the surface. The efficacy at 0.5 mg ml⁻¹ is further reduced as scrubbing velocity is increased to the level where viscous forces interfere with the integrity of the distal ends of the bristle, as shown at 0.5 mg ml⁻¹ and 48 mm s⁻¹ (FIG. 4H). As the IONP concentration is increased, scrubbing efficacy increases due to the lengthening of the bristle. Biofilm removal is significantly enhanced as IONP concentration increases to 1-2 mg ml⁻¹, achieving above 90% biofilm removal in the targeted area (FIG. 4H). Biofilm components (microbial cells and EPS matrix) were entrenched into the extended STARS bristle during the biofilm removal (FIG. 4I) and can be retrieved together with the collected bristle as a structured biohybrid complex (FIGS. 4J and 4K).

Altogether, STARS bristles operate through multiple complementary mechanisms that are capable of catalysis, physical intelligence (surface conforming, shape-adapting reconfiguration, and adjustable shear strength) and generation of tunable lateral forces and antimicrobial reagents in situ, which can explain the efficacy of biofilm disruption at multiple spatial and length scales, while entrenching biofilm components. This provides multimodal features for designing autonomous motion routines for precise biofilm treatment and sampling by combining controlled mechanical and chemical activation occurring simultaneously with topography-adaptive functionality in real-time.

Automated biofilm removal and retrieval on tooth surface: 3D-printed tooth replicas and ex vivo human teeth were employed to closely mimic the clinical conditions (FIG. 5A). Natural teeth have variations in physiological contours, geometries, and surface topography, including varying degrees of surface curvature and angles. The interdental space between teeth is a confined, hard-to-reach area where pathogenic biofilm can grow and flourish that requires manual flossing for removal. The disclosed STARS bristles can conform to these variations through their adaptive nature. The bristle's length can be reconfigured to reach distant surfaces in confined spaces as it moves from flat to curved and through interdental spaces. This adaptability to a cross-sectional model of human teeth can allow clear visualization (FIG. 5B). As bristles are swept over the interdental space, they reconfigure and conform to the curvature of the surface, transforming from a ‘brush-like shape’ to an extended ‘floss-like structure’ (FIG. 5C) that reaches the entire narrow gap. These structures dynamically change their size with a remarkable range over multiple length scales; the heights vary from submillimeter to >4.5 mm (FIG. 5D), and widths range from 3 mm to submicrometer (FIG. 5D, E), providing structural flexibility to adaptively conform to the interdental region. To evaluate motion-controlled, 3D cleaning patterns, an apparatus that enables combinations of different motions with fully automated cleaning routines were developed.

FIG. 5A shows the 3D-printed tooth mimics that were created by scanning natural human teeth for biofilm treatment and sampling analysis. FIG. 5B shows the cross-sectional model of interdental space that was developed to provide varying surface topographies. FIG. 5C shows the STARS bristles demonstrating the topographical adaptation, shape variation, and deep penetration into interdental space. FIGS. 5D and 5E show different lengths and widths of the superstructure as it adapts and accesses the interproximal space, creating a conformal flossing shape.

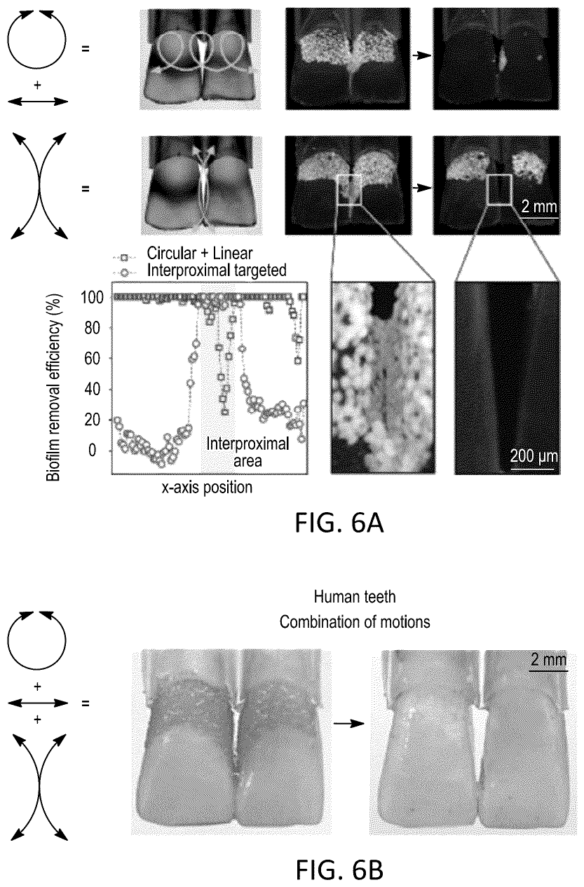

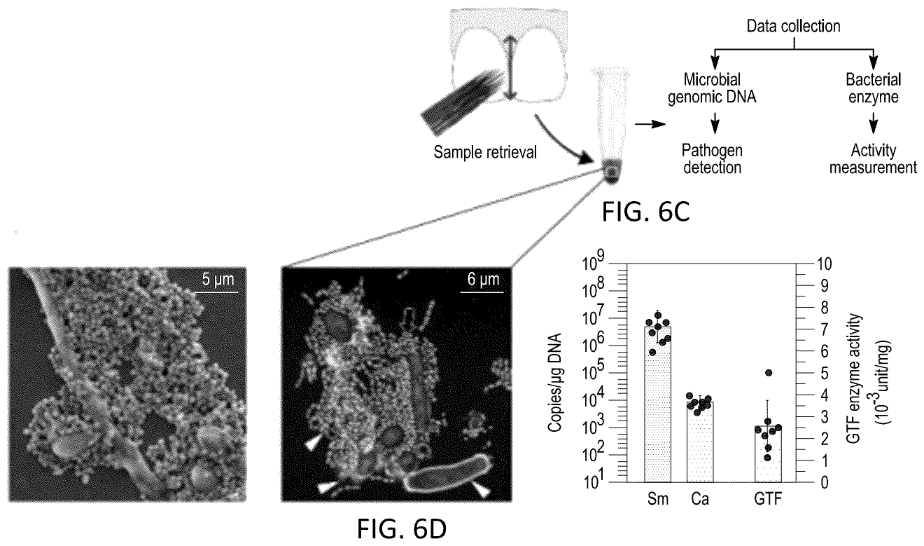

A set of fundamental STARS motion patterns, including circular, linear, and arced motions, which mimic toothbrushing and test their cleaning efficacy on 3D-printed human tooth mimics, was developed (FIG. 6A). Using a circular-linear dual motion, effective cleaning of the facial surface of anterior teeth is shown in FIG. 6A, top. However, this pattern does not effectively clean the curved interdental area in between two teeth. To address this, an arced motion was added following the contour of the interdental surface, which targeted interdental cleaning (FIG. 6A bottom). Localized biofilm removal efficacy depends on the programmed motion as well as the targeted region. The circular motion is effective in broadly cleaning the facial tooth surfaces, while the arced motion directed along the space between teeth demonstrates high interproximal removal efficacy (FIG. 6A graph). The cleaning efficacy of the combined (multi-axis) motion was assessed using an ex vivo human teeth model mimicking the natural tooth-gingival positioning and arrangement as well as its anatomical features. Complete biofilm removal from the facial and interdental spaces on human teeth by the combined STARS motions were detected (FIG. 6B).

FIG. 6A shows fundamental motion patterns that were assessed on human tooth mimics. Before and after comparisons of fluorescently labeled biofilms cleaned with a combination of circular and linear motions (top) and targeted interproximal cleaning (middle), demonstrating efficacy on complex topographies. Circular motions effectively remove the bulk of biofilm from the facial tooth surfaces, while targeted motion selectively removes biofilm from interproximal space (bottom, left). FIG. 6B shows the combined motions that were conducted for complete biofilm removal on ex vivo human teeth. FIG. 6C shows the data collection workflow using STARS to support biofilm sampling for multiplexed analysis. FIG. 6D shows an SEM image (pseudo-colored) showing intact C. albican (in cyan) and S. mutans (in green) cells entrenched by the superstructure (in gray) and the confocal image showing retrieval of biofilm components (white arrowheads) using STARS (black arrow), including bacterial cells (in green), EPS (in red), and fungal cells (outlined in blue) after targeted sampling, enabling bacterial and fungal strains identification and exoenzyme (GTF) activity analysis (graph). Sm: S. mutans, Ca: C. albicans.

As STARS continually reconfigure during biofilm cleaning at targeted areas, they incorporate biofilm contents into the bristle. This feature enables precise retrieval of biofilm contents with high spatial precision at the submillimeter level (FIG. 6A close-up). The heterogeneous distribution and uneven location of biofilms associated with human chronic infections provide a key challenge for accurate sampling, which would be particularly important for difficult-to-access biofilms such as those formed in the interdental space. To assess this capability, S. mutans and Candida albicans, which are bacterial and fungal pathogens found in virulent interkingdom biofilms on the tooth surface, were used. Furthermore, the biofilm's pathogenicity is associated with extracellular polymers (EPS)-producing exoenzyme termed glucosyltransferase (GTF), providing an opportunity to detect virulence biomarkers. After biofilm sample collection, both the microbial content and enzyme activity were analyzed (FIG. 6C). Bacteria, fungi, and EPS are entrenched within the bristles (FIG. 6D). Further analysis reveals that STARS can collect sufficient quantities of the biological materials, allowing the detection of both bacterial and fungal pathogens by qRT-PCR at expected proportions as well as quantitative measurement of GTF activity (FIG. 6D graph). The data indicate the feasibility of applying STARS for the diagnostic sampling of disease-causing biofilms, achieving localized sample retrieval for the detection of infective agents as well as their by-products and biomolecules, including polysaccharides (sugars), proteins, metabolites, and enzymes. Together, STARS enables therapeutic-diagnostic applications that combine autonomous, tether-free biofilm treatment with concomitant data collection functionality, which can provide important information supporting pathogen detection, virulence assessment, and personalized treatment of infectious diseases, and which is not easily achievable with conventional approaches.

The directed assembly of reconfigurable, cantilevered bristle-like superstructures, which are composed of packed nanoparticles by spatially and temporally modulating magnetic fields, is disclosed. These STARS, surface topography-adaptive robotic superstructures, have tunable strengths, shapes, and reactivity that are effective for removing biofilms. A spatially ordered bristle superstructure was observed with controllable stiffness that provides strong cohesion at the base where support is required and remains reconfigurable at the distal end where surface conformality is desirable. This gradient in properties allows STARS to self-support, reach, and adapt to variations in surface topography in arbitrary orientations while generating sufficient shear stress to dislodge biofilms. Furthermore, by introducing scalable and reversible architectures, the STARS length can be extended or retracted while conforming to crevices and other complex features, enabling the treatment and retrieval of distinct microbes from within biofilm on hard-to-reach surfaces with microscale precision. Notably, the catalytic property is preserved throughout reconfiguration, providing mechanochemical function afforded by the assembled superstructures.

The findings on the interdependence of superstructure conformability, topography adaptation, physical force adjustment, and spatiotemporal magnetic field control can lead to further investigations at the intersection of reconfigurable soft-matter, functional nanomaterials, and micro robotics. In addition, these dynamic structures can be actuated remotely and precisely with translational and circular motions using automated, programmable control algorithms. An automated, tetherless multi-tasking platform that integrates ‘toothbrushing-like’ and ‘flossing-like’ action simultaneously with antimicrobial activity in real-time and sample retrieval for pathogen detection was created. The disclosed feedback-guided STARS for on-demand motion patterns, control, and functional assembly can lead to highly configurable structures able to adjust to different 3D surface geometries and adhesion strengths to achieve autonomous biofilm disruption, removal, and diagnostics.

Synthesis and characterization of iron oxide nanoparticles (IONPs): The IONPs were synthesized via a facile solvothermal method. 0.82 g of iron (III) chloride was dissolved into 40 ml of ethylene glycol. Then, 3.6 g of sodium acetate was added to the solution under vigorous stirring at room temperature for 30 min. Subsequently, the mixture was transferred to a custom-built 50 ml Teflon-lined stainless-steel autoclave and heated for 12 h. After the autoclave was cooled to room temperature naturally, the IONP precipitate was collected, rinsed several times with ethanol, and then dried at 60° C. for 3 h. The synthesized IONPs were characterized using scanning electron microscopy (SEM, FEI Quanta 600, FEI, Portland, OR, USA). For IONPs size distribution, the region of IONPs was automatically detected and characterized by the size invariant circle detection method implemented in MATLAB built-in function “imfindcircles.”

Magnetic field control device: The magnetic fields are controlled with a device combining programmable electromagnets and servo motors. The magnetic field for the reconfigurable STARS bristle was formed by two coaxially arranged electromagnets at a distance of 8 mm. Between the two electromagnets, an actuation vessel with the dimension of 15×6.7×13 mm³ (width×depth×height, about 1.3 ml) was designed to accommodate IONPs suspension, and a holder was constructed to maintain an 8 mm gap between the two electromagnets. Both the electromagnets and servo motors were mounted around the actuation vessel and remained fixed in the same position for all experiments. A ferrite core with a diameter of 10 mm was situated in the center of the rear electromagnet. An iron core with a diameter of 2 mm was positioned in the center of the forward electromagnet. The position of the forward iron core is moved in varying trajectories using servo motors.

For side-to-side motion, a micro servo motor (SG90) was programmed to move 50 degrees (between 65 and 115 degrees) with an arm of 14 mm in length. A symmetrical arc-shaped movement of approximately 12 mm was implemented with various linear velocities from 6 to 48 mm s⁻¹. For circular motion, a continuous rotation servo motor (FS90R) was used and programmed to allow unrestricted movement at angles using a relatively short arm of 2 mm in length. A continuous circular movement was generated centered with the axis of electromagnets with a linear velocity 12 mm s⁻¹ in clockwise or counterclockwise directions.

To create multimodal motions with a high degree of repeatability in position and timing, a sliding vessel was created. The body of the device consisted of two different parts, a holder for the electromagnets and servos and a sliding vessel. One servo drives a pinion gear attached to the rack of the sliding vessel for left-to-right motion, and the other servo drives the forward iron core for circular or arc-shaped motions. The sliding vessel held the tooth replicas and natural teeth and was actuated by a programmable micro servo motor. The motion of the sliding vessel was coupled with the programmable motion of the electromagnet iron core, which allowed a combination of motions to mimic brushing patterns.

The high-gradient magnetic field was directed by the repositioning of the iron core, and the various positions were mainly implemented by the movement of the servo controlling the iron core within the forward electromagnet (side to side and circular by regular and continuous micro servo), during which the magnetic field remained constant. Actuation in all experiments was performed via the application of a magnetic field and servo programmed to achieve the desired motion pattern. The movement of the micro servo arm and the magnetic fields of electromagnets were controlled using a microcontroller (Arduino Nano, Arduino, Somerville, MA, USA). Each electromagnet was energized independently using a programmable DC power supply (Sorensen XTR60-14, AMETEK Programmable Power, San Diego, USA) and Arduino integrated development environment (IDE) software for system coding.

The geometries of the actuation vessel and the body of the device were developed and designed with Onshape Computer-Aided Design (CAD) software (Onshape Inc, Cambridge, USA). The actuation vessel and the body of the device were fabricated using low force stereolithography (SLA) 3D printer (Form 3B, Formlabs Inc., MA, USA) with 25×25 μm² lateral resolution and 50 μm layer thickness with a biocompatible photopolymer resin (Dental SG V1 resin, Formlabs Inc., MA, USA). For 3D printed parts, rinsing was performed for 20 min in fresh 99% isopropanol (IPA), air drying for 30 min and 60 min post-print ultraviolet light curing (405 nm light at 60° C.) (FormCure, Formlabs Inc., MA, USA).

Bristle-like STARS formation: The bristle-like superstructures were assembled using a magnetic field control system as described above. The dispersed IONPs were collected by magnetic fields from the forward electromagnet for 5 sec forming a rounded dome shape, and the strength of the magnetic field used for the standardized biofilm removal experiments was measured to be 90 and 25 mT (MF100; Extech Instruments, Boston, MA, USA) at the forward and rear regions, respectively. The STARS were then designed to orthogonally extend from the forward vessel wall to form elongated bristle-like structures for 25 sec by energizing the rear electromagnet. When moving the bristles from the front to the rear, the strength of the magnetic field was measured to be 35 and 70 mT at the forward and rear regions, respectively. At the beginning of the next cycle, IONPs were recollected in the forward region. A cycle of 30 seconds was repeated up to 20 times during dental biofilm removal experiments. The appearance and physical/mechanical properties of STARS bristles can be precisely controlled by the concentration of IONPs (0.5 to 2.0 mg ml⁻¹), the sweep velocity of the servo (6 to 48 mm s⁻¹), and the strength of magnetic fields (13.3 to 96.4 mT) at the center of the actuation vessel.

The STARS bristle formation and movement videos in the actuation vessel were captured using a Zeiss Axio Zoom.V16 fluorescence upright stereo zoom microscope system (Carl Zeiss Microscopy GmbH, Jena, Germany) with a 1× objective (numerical aperture, 0.25) at a video frame rate of up to 100 fps. The videos were processed using Zeiss Zen Blue software to determine the position, velocity, and length of STARS bristle.

Surface conforming property of STARS bristle: To evaluate the reconfigurability and adaptation of STARS bristles, surfaces with various topographies, including repeated circular, square, and triangular patterns, were designed with Onshape. All patterned surfaces repeat the unit structure four times within an 8.5 mm width, and the depth of each unit structure is 0.9 mm. The patterned surfaces were 3D printed using a Formlabs 3B printer with Dental SG V1 resin, as described above. The patterned surfaces were suspended in the vessel at 2.2 mm distance from the forward vessel wall, and a STARS bristle (2 mg ml⁻¹) was formed and moved using the field control methods described above.

Force measurements of STARS bristle: Molds for force-sensing microcantilevers with a square cross-section were created using the Formlabs 3B printer with Dental SG V1 resin as described above. After washing and curing, the molds were coated with a separator (Gingifast Separator, Zhermack, Italy). Molds were filled with polydimethylsiloxane (PDMS, Sylgard 184, Dow Corning Corporation, Midland, MI, USA) elastomers and degassed. The excess PDMS was removed from the surface by scraping. After oven curing at 70° C. for 2 h, cantilevers were individually removed and connected to the arm of a 4-axis nano manipulator (MX7600, Siskiyou Corporation, Grants Pass, OR) to control the position of the PDMS microcantilever. The cohesion force and shear stress of STARS bristle were measured based on the relationship between the elastic deflection of the end-loaded cantilever beam and the applied force by the STARS bristle.

To estimate the shear stress exerted by STARS bristles, PDMS micropillars with a high aspect ratio were fabricated out of a silicon hard master. Briefly, 4 μm S1805 positive photoresist (Microposit, Philadelphia, USA) was spray-coated onto a silicon wafer and patterned through standard photolithography processes. The wafer was then etched in deep reactive ion etching (DRIE, SPTS Rapier Si DRIE) to create microwells with desired heights for PDMS molding. The etching depth was 240 μm, while the diameter was maintained to be the same. The wafer was then cleaned sequentially in nanostrip, deionized water, acetone, isopropyl alcohol (IPA) and deionized water again. PDMS monomer and crosslinker were mixed with a ratio of 10:1 and poured on top of the dried silicon wafer. The mixture was degassed and cured at 70° C. in an oven for 2 h. To facilitate the demolding process, Trichloro (1H,1H,2H,2H-perfluorooctyl) silane (97%, Sigma-Aldrich) was vapor coated in a vacuum chamber for 15 min, prior to PDMS pouring. The cured PDMS micropillars were peeled off from the silicon hard master. The micropillars were cut-off individually under the fluorescence upright stereo zoom microscope (Zeiss Axio Zoom.V16, Carl Zeiss Microscopy GmbH, Jena, Germany) and attached to the 3D printed holder for shear stress measurement. The maximum elastic deflection of the end-loaded micropillars was measured while sweeping STARS bristles laterally across the micropillar tip.

The force measurement videos in the actuation vessel were recorded using a Zeiss Axio Zoom.V16 fluorescence upright stereo zoom microscope system with a 1× objective (numerical aperture, 0.25) at a video frame rate of about 100 fps. The captured frames were analyzed from each video in ImageJ to measure the deflection.

Catalytic activity of STARS bristles: The peroxidase-like activity was tested via an established colorimetric method using 3,3′,5,5′-tetramethylbenzidine (TMB) as a substrate which generates a blue color with specific absorption at 652 nm after reacting with free radicals catalyzed by IONP in the presence of hydrogen peroxide over time. Briefly, the bristles were formed at different concentrations (0.5, 1 and 2 mg ml⁻¹) of IONPs under 12 mm s⁻¹ of sweep velocity and 69 mT of magnetic fields. The reaction mixture of 1 mL sodium acetate (NaOAc) buffer (0.1 M, pH 5.5) containing 0.1% H₂O₂ and 100 g of TMB was incubated at room temperature during the actuation and assayed spectrophotometrically. The time-lapse reactions were monitored at 652 nm using a Beckman DU800 spectrophotometer (Beckman Coulter, Inc., Fullerton, CA, USA).

Biofilm formation on model surfaces and human teeth: Individual slabs were created as model surfaces with dimensions of 4.0×2.2×4.0 mm³. A T-shaped mechanical locking structure was implemented for secure holding inside the actuation vessel. The 3D structure of the slabs was located where the highest magnetic flux density was controlled inside the actuation vessel. The 3D printed slabs were polished using a muslin wheel (Kerr Dental, USA) and fine pumice (Benco, USA). After polishing, they were bath sonicated for 5 min to wash off the pumice.

Human incisor teeth extracted in the dental clinic (for surgical reasons) at the School of Dental Medicine were collected and repurposed for this study without any identifiers. The teeth were matched into pairs based on morphology and dimensions. Tooth pairs were scanned using CEREC Omnicam Intraoral Scanner and integrated CEREC 5.0 software (Dentsply Sirona, USA) to implement a tooth mimic model. The scanned high-resolution 3D CAD images were used to generate 3D printed teeth replicas and design a custom 3D printed holder to mimic the gingival margins of the natural teeth using 3D CAD software (Blender, version 2.91.0; Blender Foundation, Amsterdam, Netherlands). 3D printing was done using Formlabs 3B printer with a biocompatible Dental SG V1 resin, and the 3D printed teeth replicas were polished as described above. The polished 3D printed tooth pairs and the natural tooth pairs were sterilized in an autoclave for 20 min at 121° C., placed in the custom-fit holder, and subsequently used for biofilm formation. The biofilms formed on teeth were placed in the actuation vessel and subjected to STARS bristle treatment.

Biofilms were formed on saliva-coated 3D printed slabs, 3D printed tooth pairs or ex vivo human tooth pairs. Streptococcus mutans UA159, a biofilm-forming model oral pathogen, was grown in ultrafiltered (10-kDa cut-off; Millipore, MA, USA) tryptone-yeast extract (UFTYE) broth containing 1% (w/v) glucose at 37° C. and 5% CO₂ to mid-exponential phase. Both saliva-coated 3D printed slab and tooth pairs were mounted vertically in 24-well plates and inoculated with ˜2×10⁵ colony-forming units (CFU) of actively growing S. mutans cells per milliliter in UFTYE containing 1% (w/v) sucrose at 37° C. with 5% CO₂ for 43 h. The culture medium was changed twice daily (at 19 and 29 h) until the end of the experimental period (43 h). In a separate experiment, Candida albicans SC5314 (a well-characterized opportunistic fungal pathogen) was used to generate mixed-species biofilms with S. mutans. For inoculum preparation, C. albicans (yeast form) and S. mutans cells were grown in ultrafiltered UFTYE broth (at pH 5.5 and pH 7.0 for C. albicans and S. mutans, respectively) containing 1% (w/v) glucose at 37° C. and 5% CO₂ to mid-exponential phase. Saliva-coated 3D printed slabs were mounted vertically in 24-well plates and inoculated with ˜2×10⁵ colony-forming units (CFU) of actively growing S. mutans cells and ˜2×10⁴ (CFU ml⁻¹) of C. albicans (yeast cells) per milliliter in UFTYE containing 1% (w/v) sucrose at 37° C. with 5% CO₂ for 43 h.

Biofilm disruption and removal: Biofilms were treated with STARS bristles assembled from IONPs (0.5, 1, and 2 mg ml⁻¹ final concentration) solution in the actuation vessel as described above. Customized IONP formulation was used to enhance the catalytic bioactivity with EPS-degrading enzymes (dextranase/mutanase, 25/5 U) and 1% H₂O₂ (1% v/v). Briefly, biofilms were placed in the actuation vessel containing IONP solution for 10 min followed by the addition of 1% H₂O₂ and subsequent modulation of the magnetic field, which forms STARS bristles. The velocity, trajectories, and motion frequencies were controlled as described in the previous sections. Biofilm removal in all experiments was performed via the application of an external magnetic field and servo movement preprogrammed to achieve the desired trajectories, velocities, and motion patterns.

Standard culturing method (number of viable cells by CFU determination) and stereoscope-based fluorescence imaging were performed to assess the biofilm removal by the STARS bristles. For the culturing method, the total number of CFU per biofilm was determined after biofilm treatment. Briefly, the removed biofilm was collected and homogenized via water bath sonication followed by probe sonication (30 sec pulse at an output of 7 W; Branson Sonifier 150; Branson Ultrasonics, CT, USA). Homogenized biofilm suspensions were serially diluted and subjected to microbiological analysis. The total number of viable cells was determined by CFU counting. For fluorescence imaging, SYTO9 (485/498 nm; Molecular Probes, Carlsbad, CA, USA) fluorescent probe was used for labeling bacterial cells, and Alexa Fluor 647 dextran conjugate (647/668 nm; Molecular Probes, Carlsbad, CA, USA) was used for labeling EPS. Before and after removal, images of the samples were taken with the Zeiss Axio Zoom.V16 fluorescence upright stereo zoom microscope system (Carl Zeiss Microscopy GmbH, Jena, Germany) with 1× objective (numerical aperture, 0.25).

To analyze the area of biofilms on the slab, open-source Fiji software was used. Images were processed for the green channel (SYTO9) due to significant cross-talk from the red channel (Alexa Fluor 647) from stereo microscopy. A standardized and constant rectangular-shaped region of interest (ROI; 4,000×2,000 μm², width and height) was selected to calculate and compare only the biofilm area on the 4×2 mm² substrate. A median filter (radius=2) was applied to eliminate noise and reduce false segmentation of background and out-of-focus signals. Otsu's automatic global thresholding algorithm featured in Fiji was used to classify and segment the colonized area of biofilms on the ROIs. The biofilm removal efficacy was calculated as (a-b)/a, where “a” was the segmented area of biofilm before treatment, and “b” was the segmented area of biofilm after treatment for comparing the removal efficacy of biofilms under the various actuating conditions as the normalized area of biofilm.

Biofilm entrenchment by STARS and visualization: Super-resolution confocal microscopy was performed to visualize the biofilm components (bacteria, fungi, and EPS) that become entrenched among the extended STARS bristles. The EPS glucan matrix was labeled with Alexa Fluor 647 dextran conjugate (Molecular Probes). S. mutans and C. albicans (if applicable) were stained with SYTO9 (Molecular Probes) and Concanavalin A-tetramethylrhodamine conjugate (Molecular Probes), respectively. After biofilm removal, STARS bristles were collected and immobilized in 1% agarose for super-resolution imaging using a 40× water immersion objective (numerical aperture=1.2) on an upright confocal microscope (Carl Zeiss LSM 800, Germany) with Airyscan. The STARS bristles were visualized using the reflection mode and a 405 nm laser. In a separate experiment, biofilm components entrenched in STARS bristles were dehydrated through a graded ethanol series, and examined by scanning electron microscope (SEM, FEI Quanta 600, FEI, Portland, OR, USA).

Analysis of the biofilm retrieval components: STARS bristles with entrenched biofilm components were subject to molecular and enzymatic analyses after retrieval. The fungal and bacterial genomic DNA was extracted and purified from the sample using DNeasy PowerLyzer Microbial Kit (Qiagen, Germany), following the manufacturer's instructions. The qPCR reaction was performed using a CFX96 Real-Time PCR Detection System (Bio-Rad Laboratories, Hercules, CA, USA) and PowerTrack SYBR Green Master Mix (ThermoFisher, Waltham, MA, USA). Specific primers were used to detect each microbial species (S. mutans, forward: 5′-ACCAGAAAGGGACGGCTAAC-3′, reverse: 5′-TAGCCTTTTACTCCAGACTTTCCTG-3′; C. albicans, forward: 5′-AGAACGATAATAACGACGATGA-3′, reverse: 5′-AGTCATTGTAGTAATCCATCTCA-3′).

Bacterial glucosyltransferase (Gtf) activity was analyzed using scintillation counting. The Gtf activity in the sample was measured in terms of incorporation of [¹⁴C] glucose from radiolabeled sucrose substrate (New England Nuclear Research Products, Boston, MA) into the glucan product for 4 h at 37° C. One unit (U) of Gtf enzyme was defined as the amount of enzyme needed to incorporate 1 mol glucose into glucan over a 4 h reaction period.

Statistical analysis: All statistical analyses were performed with GraphPad Prism 8.0 (GraphPad Software, Inc., La Jolla, CA). All data are represented as mean±s.d. Comparisons between multiple groups were performed using a two-sided one-way analysis of variance (ANOVA) with post-hoc Tukey's test, where P<0.05 was considered significant and P>0.05 was considered not significant. At least three independent experiments were performed unless otherwise stated.

### Example 2: Microrobotics for Precision Biofilm Diagnostics and Treatment

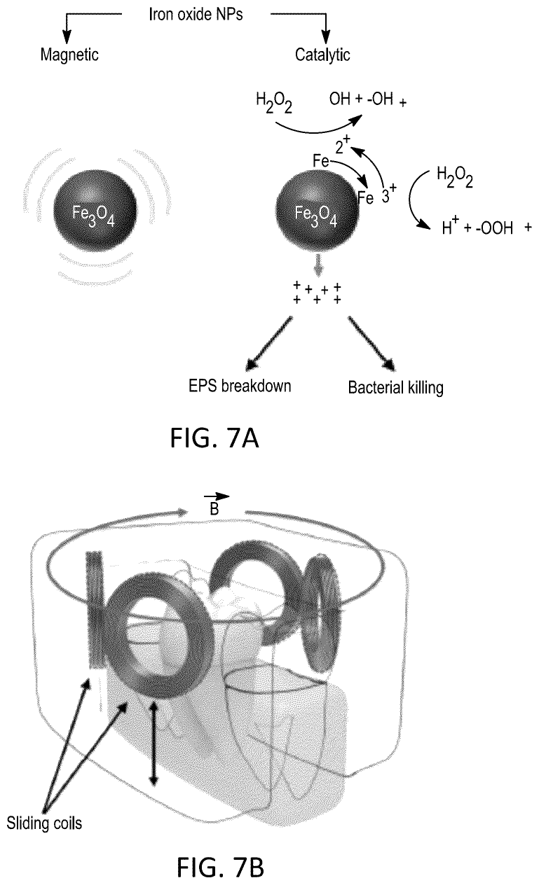

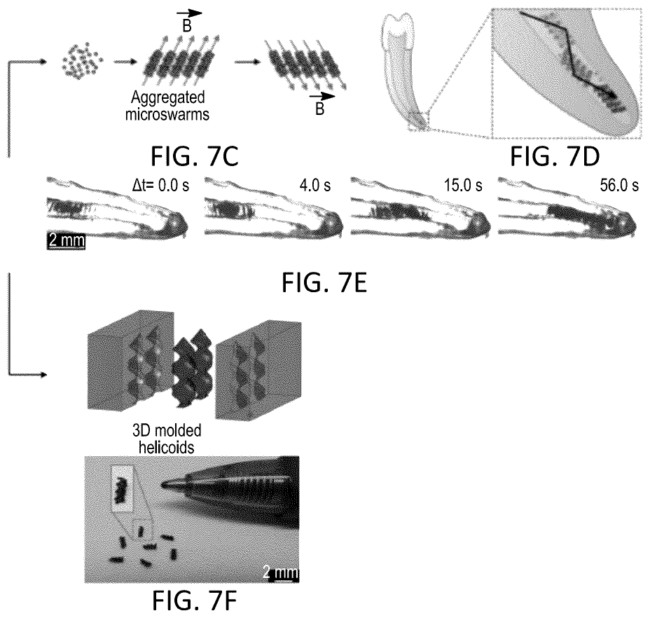

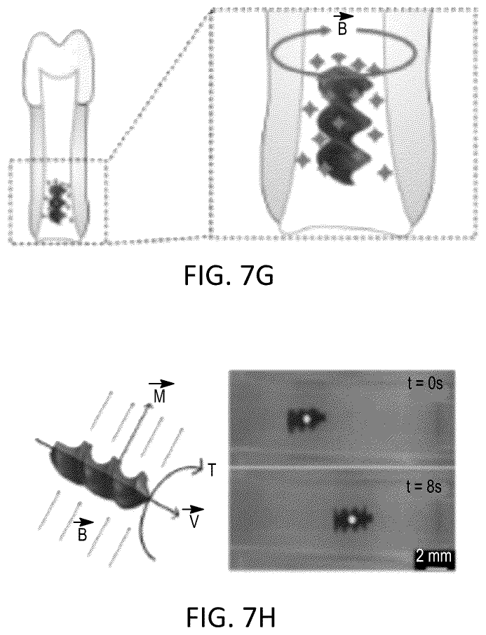

Recent advances in nanotechnology and robotics have enabled the integration of magnetocatalytic nanoparticles with microrobotics principles to target and eradicate biofilms in clinically challenging settings. Here, the catalytic and magnetic properties of iron oxide nanoparticles (NPs) were used to introduce a microrobotics platform designed for biofilm treatment and diagnostics (FIG. 7a) using endodontic biofilm models as an exemplar proof-of-concept application.

Targeting endodontic biofilms. Incomplete root canal disinfection remains the main cause of treatment failure leading to persistent endodontic infections and apical periodontitis in conventional antimicrobial procedures. The anatomical complexities in the root canal system hinder the effective removal of biofilms, while approaches to diagnose or assess disinfection efficacy are limited. New disinfection modalities such as photon-induced, photoacoustic streaming, passive ultrasonic irrigation, antimicrobial nanoparticles and photodynamic therapy have been proposed and or used for endodontic biofilm treatment. However, these approaches lack controlled targeting of the anatomical complexities and are unable to retrieve biofilm samples for diagnostics. New technologies can enable multifunctionality to access difficult-to-reach surfaces and perform biofilm removal, and microbial detection simultaneously for effective and precise endodontic therapy. Such multimodal approaches can be achieved using microrobotics.

Magnetically driven catalytic antibiofilm robots. Iron oxide NPs were used as the fundamental building blocks to create distinctive microrobots to kill, degrade, and retrieve biofilms from confined spaces found in the root canal system, a challenging anatomical space in the oral cavity. The average diameter of the iron oxide nanoparticles is estimated to be 464.9 nm with a standard deviation of 36.08 nm. Iron oxide NPs have catalytic properties that arise from intrinsic enzyme-like (peroxidase-like) activity that activates H₂O₂ to generate bioactive molecules in situ to disrupt oral biofilms (FIG. 7. a). Furthermore, iron oxide NPs are widely used in nanomedicine due to their minimal cytotoxicity, excellent physicochemical properties, stability in aqueous solutions and biocompatibility. One of the first nanoparticle formulations to be approved by the Food and Drug Administration (FDA) for clinical use was an iron oxide nanoparticle contrast agent for magnetic resonance imaging (Feridex®), while another similar preparation (Feraheme®) was subsequently approved for the treatment of iron deficiency anemia. The histopathological analysis of gingival, mucosal and other tissues, including major organs such as liver and kidney, showed no signs of harmful effects, such as proliferative changes, inflammatory responses, or necrosis, indicating high histocompatibility of both in-house and FDA approved IONP formulations with H₂O₂ treatment. Furthermore, NPs can be directed to precise locations using magnetic fields that are generated by permanent magnets or electromagnets. Magnetic actuation allows tether-free controlled motion, enables a wide variety of robotic locomotion strategies and can readily and harmlessly penetrate biological and synthetic materials and direct robots' motion in confined spaces (FIG. 7.b).

The flexibility of ‘NPs building blocks’ allows the formation of reconfigurable aggregated structures, inclusion in 3D molded soft helicoids or even direct 3D printing. Here, two platforms were developed. The first system uses magnetic forces to concentrate NPs and form structured aggregates (FIG. 7. c). These aggregated structures are catalytically active robotic structures that are driven by controlled magnetic fields to the apical region of the tooth (FIG. 7.d, e). They are magnetically driven to mechanically remove and retrieve the disrupted biofilms. The second platform is designed to be amenable to micromolding with 3D printing techniques to create specific shapes for intracanal locomotion. Miniaturized, helical robots are fabricated from a matrix of biocompatible hydrogel within which NPs are embedded. The robot shape features a double helix having 1.5 turns swept around a central axis. The molds are fabricated using a stereolithography 3D printer (FIG. 7. f). Navigation of microrobots is controlled by rotating magnetic fields generated by electromagnets to propel through fluids in the desired direction (FIG. 7. h). 3D molded helicoids guided to the apical region can be used to transport bioactive or drugs and release them on-site, i.e., apical region (FIG. 7. g), which can lead to multi-purpose applications.

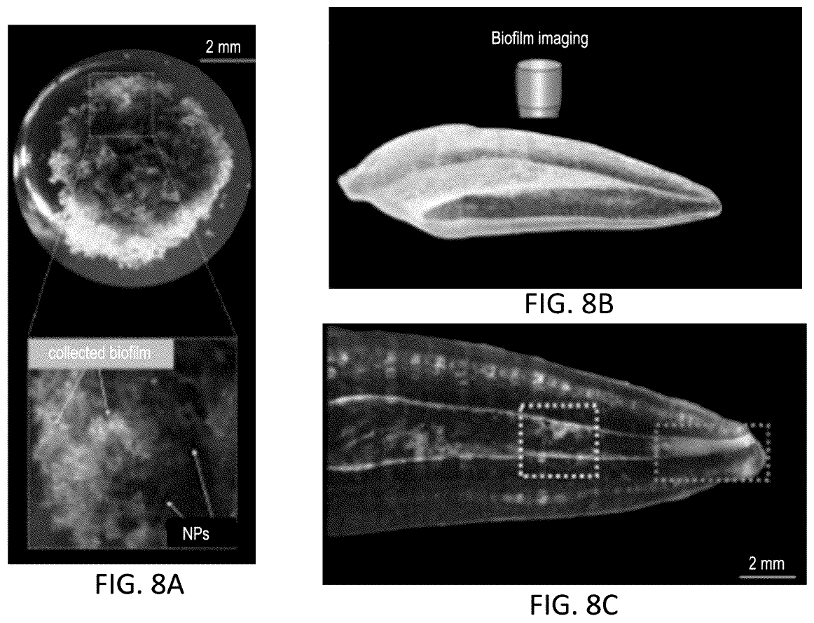

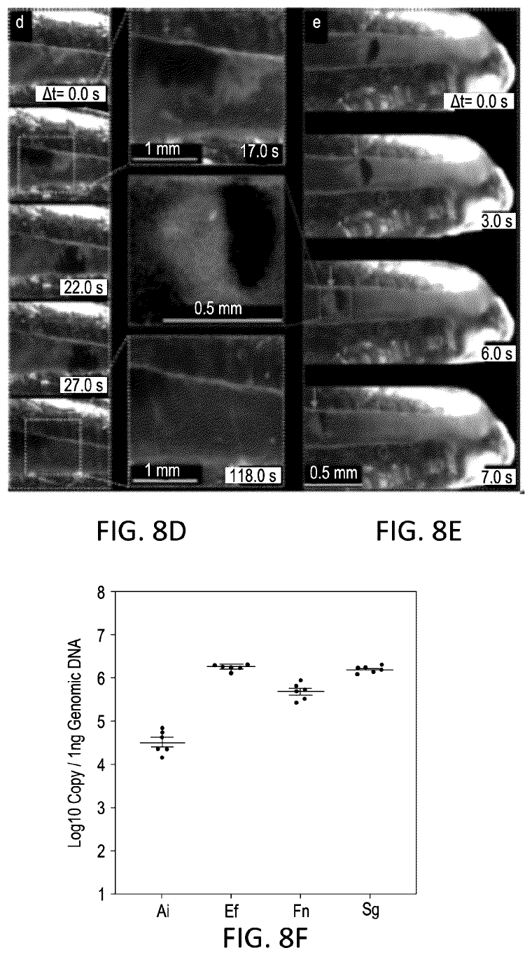

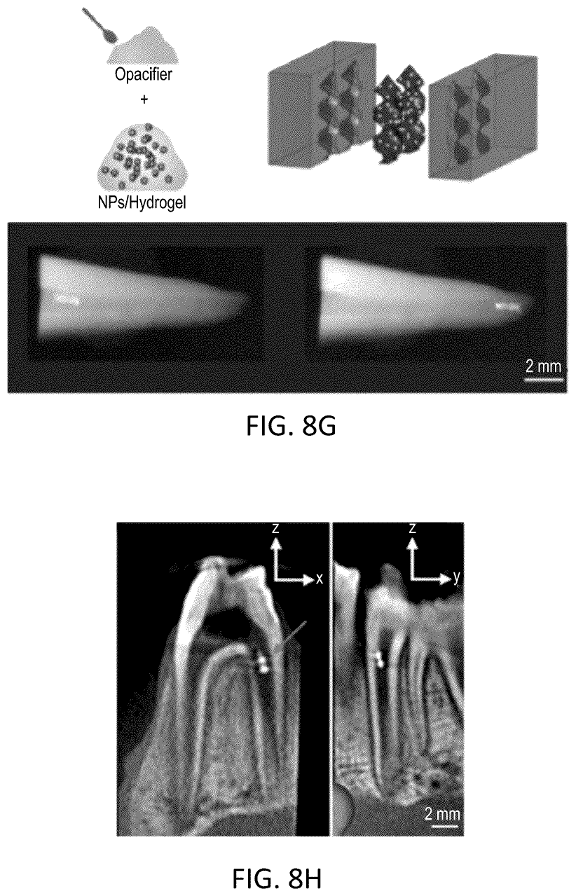

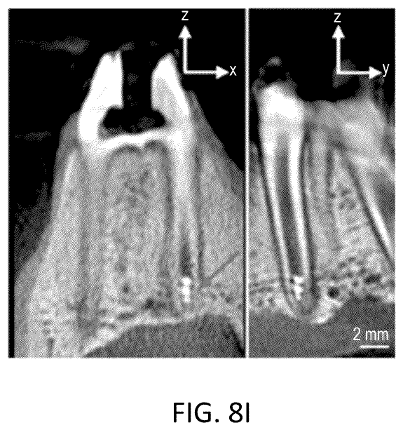

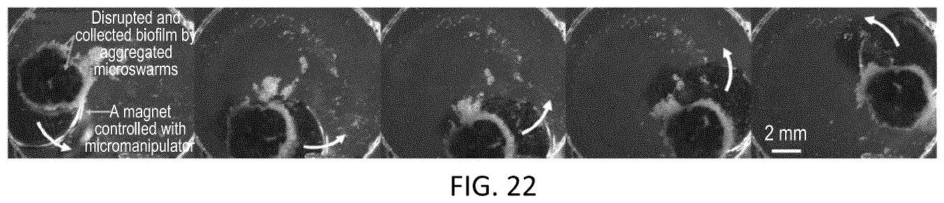

Aggregated structures. Iron oxide NPs can reversibly form aggregated structures by applying and removing the external magnetic fields on-demand. Once the aggregates are formed, collective behavior can be achieved through precise control over the magnetic fields. Functioning as a dynamic collective unit under the magnetic field control, aggregated structures can perform complex tasks and can reconfigure and adapt to the confined space. Using 3D printed teeth replicas generated based on microCT scans of natural human teeth, the data show that the aggregated structures can be moved and directed by the magnetic field and adapt to the variable canal geometry to reach the apical region. The size of the aggregates between the canal walls had a wide range of sizes (from 154.5 μm to 844.4 μm). By measuring the structures' width in a corono-apical direction, the range was between 37.8 μm and 1235.9 μm. (FIG. 7. e). To demonstrate the ability of aggregated structures to disrupt the biofilm and retrieve biofilm samples, fluorescently labeled (SYTO9) 72 h mixed-species biofilms containing S. gordonii, E. faecalis, F. nucleatum, and A. israelii (initial inoculum size ˜2×10⁵, ˜2×10⁵, ˜2×10⁸ and ˜1×10⁸ respectively, grown anaerobically in BHI media supplemented with 5 mg/ml yeast extract, 0.5 mg/ml L-Cysteine HCl, 5 μg/ml hemin and 0.5 μg/ml vitamin K1), were prepared on a glass surface. The NPs (1-2 mg/ml) were introduced near the biofilm surface and catalytically activated by adding 3% H₂O₂ for bacterial disruption/EPS degradation, followed by magnetic actuation. Suspended NPs readily formed aggregated assemblies when actuated using a neodymium iron boron magnet (diameter=6 mm). In addition, as the structures mechanically disrupt biofilms, bacterial cells become bound to the aggregated NPs (FIG. 8. A and FIG. 22). This indicates that the aggregated structures can be used to retrieve biofilms for the purpose of sampling.

To test the ability of the structures to disrupt and retrieve samples from the apical regions, 14 day mixed-species biofilms (same bacterial species and inoculum size as described above) were prepared inside vertically positioned 3D printed teeth replicas (media was changed after 24 h, then after every 48 h) (FIG. 8. b,c). The NP suspension was introduced in the coronal aspect of the canal (approximately 0.5 mg/ml). The structures were actuated and precisely controlled using an electromagnetic apparatus (FIG. 7.b) that generates a transverse magnetic field (60 mT) using coaxially positioned electromagnets with iron cores, allowing them to move in a coronal and apical direction to disrupt biofilm inside the canal. The aggregated microswarm had an average size of 797.3 μm with a standard deviation of 153.12 μm between the canal walls and an average size of 1196.6 μm with a standard deviation of 92.1 μm along corono-apical direction (FIG. 8.d). Moreover, aggregated structures successfully retrieved a portion of the apical biofilm samples for analysis. The size of the aggregated microswarm retrieving a biofilm sample from the apical region in FIG. 2. e was 553.4 μm between the canal walls and 165.9 μm corono-apically (FIG. 8. e). The retrieved biofilms were collected and subjected to standard qPCR analysis (FIG. 8. f). All four species were readily detected using species-specific probes. In addition, all nanoparticles appeared to be removed based on visualization using a stereo-zoom microscope. However, elemental analyses such as inductively coupled plasma-optical emission spectrometry and scanning electron microscopy with energy dispersive spectroscopy are needed to further assess nanoparticle removal.

3D molded helicoids. Given the flexibility of the iron oxide NPs as building blocks, a molded robotic system was developed using 1-mm negative molds designed in SolidWorks and 3D printed using a ProJet 6000 HD stereolithography printer (3D Systems, Rock Hill, SC, USA). Helicoid 3D molded robots (two helices wrapped around a central axis) are formed by embedding NPs within a thermo-reversible granulated agar gel (Difco, BD Biosciences, Sparks, MD, USA) in a final soft robot composition that contains 25% glycerol, 3% (w/v) agar gel within which 10% NPs are embedded. The NP-embedded gel is used to fill the negative molds to create 3D molded soft helicoids with a size of ˜700 μm by 1.5 mm. When directed by the magnetic fields, the 3D molded helicoids can propel with high efficacy (FIG. 7. h). When a magnetic torque is applied, a 3D molded helicoid rotates around its central axis. This results in forwarding locomotion due to the chiral geometry and corkscrew-like action of the helicoid. Notably, 3D molded helicoid can be cargo loaded (i.e., drugs) to achieve targeted drug delivery through on-demand drug release. The loading and releasing of drugs from microrobots have been shown to be feasible using different approaches. For example, the cargo can be encapsulated in protective microcapsules produced via glass capillary microfluidics using double emulsions. Drugs loaded within the microrobots can be released on-demand by the application of rapid oscillation of a magnetic field. Here, the helicoid was loaded with a chemical (opacifier) to address an important application hurdle while showing the feasibility of incorporating additional substances into the robotic structure.

A practical challenge facing microrobotics is the difficulty of tracking inside the body. Real-time updates of position would aid precise navigation systems, ensure targeting, and confirm that microrobots are performing their desired function. To address the challenge of real-time tracking and locating the microrobots, bismuth oxide opacifiers are incorporated 3% (w/v) within the matrix of 3D molded helicoids to enhance their radiopacity (FIG. 8. g top panel). X-ray images using an intraoral radiographic sensor and a dental X-ray unit were obtained and showed the radiopaque 3D helicoids within the canal of the extracted tooth (FIG. 8. G bottom panel). Importantly, the feasibility of magnetic actuation of 3D molded helicoids in an ex vivo model of an intact pig jaw model (Sierra for Medical Science, Inc.) uninterrupted by the soft and hard tissues surrounding the teeth (intact periodontium) was shown. In addition, the controlled movement of the robots from the coronal to the apical region within the canal was also shown, as imaged with cone beam computed tomography (CBCT) (FIG. 8. h, i), demonstrating the clinical feasibility of intracanal imaging of microrobots. The permeability of the hydrogel allows for the diffusion of H₂O₂ to react with embedded NPs to release reactive oxygen species (ROS). This property, together with the ability of 3D microrobots to controllably propel through fluids inside the canal, opens exciting opportunities for both biofilm disruption and drug delivery in situ.

The multiple functions demonstrated above can also lead to new methods for biofilm treatment and disinfection in regenerative endodontics, where mechanical instrumentation is not desirable and treatment relies mainly on the chemical effect of antimicrobials. The 3D molded soft helicoids can be precisely controlled and actuated within the root canal space of incompletely formed roots to deliver bioactive on-site to achieve chemical and mechanical disruption of biofilm, while being tracked and located with available radiographic imaging techniques. Notably, 3D molded helicoids can be loaded with microcapsules that contain a clinically used antibiotic mixture (i.e., metronidazole and ciprofloxacin). The cargo (antibiotics) can be released on-demand via the application of rapid oscillation of the magnetic field.

A new technique for precision biofilm treatment using microrobotic platforms with potential for both diagnostics and therapeutics in the root canal system, a clinically challenging anatomical space in the oral cavity is disclosed. The feasibility of tetherless magnetic control uninterrupted by the presence of the intact periodontium surrounding the tooth is shown. Iron oxide nanoparticles were used as fundamental building blocks, which enables significant design flexibility. NPs can form aggregated structures by controlled magnetic fields, which are capable of navigating inside the tooth canal and simultaneously disrupting biofilms and retrieving biological information for pathogen detection using microbiological DNA analysis. Alternatively, NPs can be incorporated into 3D molded soft helicoids that are precisely driven to the apical third of the canal. In addition, the incorporation of opacifiers into the helicoids provides opportunities for real-time tracking using existing radiographic imaging. The inclusion of additional chemicals can provide for drug loading, transport, and on-site released in a controllable manner.

The disclosed micro/nanorobots can be precisely guided to reach the apical area and used to deliver bioactive or drugs in situ to achieve both diagnostics and chemical disinfection with tissue regeneration. The disclosed subject matter can be used in various fields.

### Example 3: Nanozyme-Based Robotics Approach for Targeting Fungal Infection

The disclosed subject matter provides microrobotic assemblies, including nanozymes using fine-scale spatiotemporal control for localized catalysis and precise targeting of infection sites to achieve in situ fungal capturing. Using electromagnetic field frequency and positioning modulation, morphology-adjustable nanozyme microrobots that can freely extend, retract, roll, vibrate and glide with high spatiotemporal precision were assembled. The catalytic activity of these nanozyme assemblies varies depending on the motion, velocity and shape, providing on-site reactive oxygen species (ROS) generation. The nanozyme assemblies unexpectedly bind avidly to fungal cells (C. albicans) and, more specifically than to human (gingival) cells. This selective binding interaction to fungi can further promote localized nanozyme targeting in situ. The directed targeting of fungal cells through cell-nanozyme binding using cell spheroid and tissue infection models were shown. The nanozyme-based robotics technique resulted in complete C. albicans eradications, providing a uniquely effective and targeted antifungal modality for pathogen control at the infection site.

### Results and Discussion

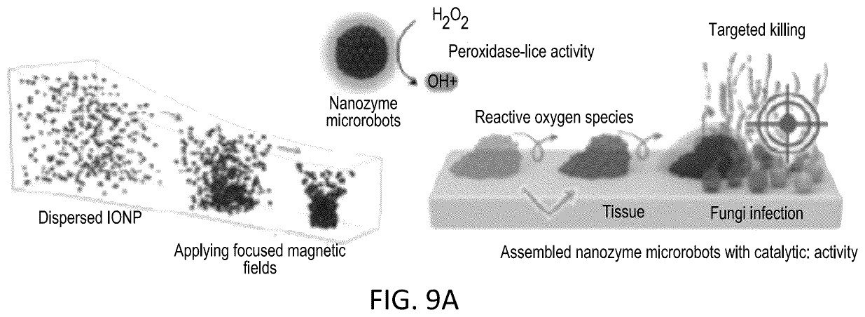

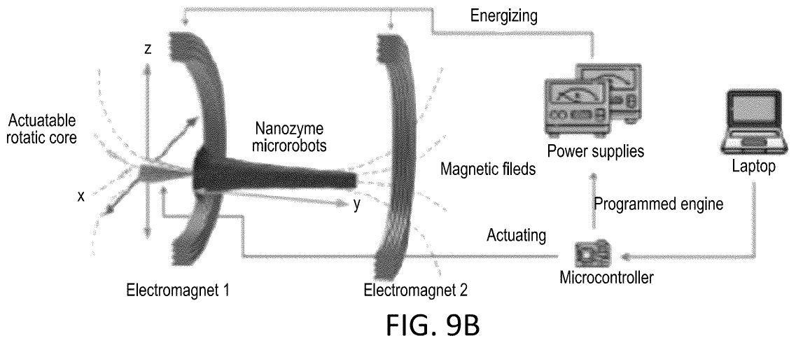

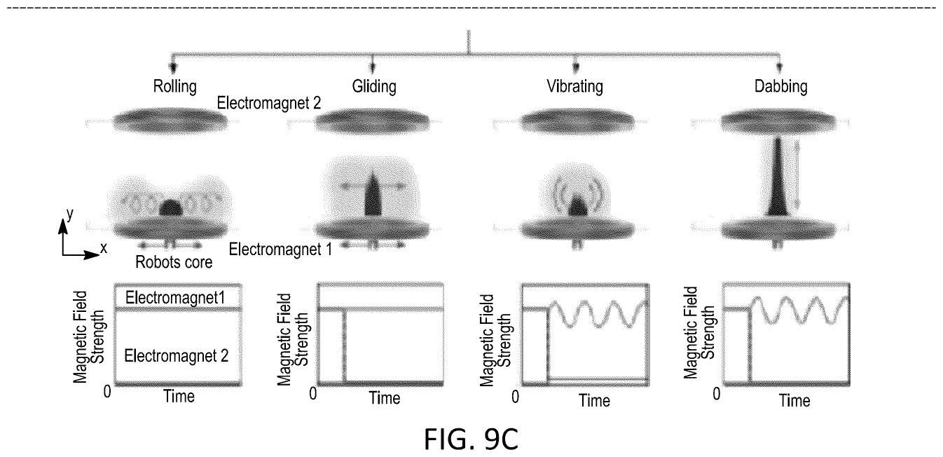

Preparation and assembly of nanozyme microrobotic assemblies: FIG. 9 shows the efficient catalysis of iron oxide nanoparticles (termed nanozymes) and their magnetic properties to create shape-tunable assemblies using electromagnetic field frequency modulation and fine-scale spatiotemporal positioning while preserving the catalytic activity. Nanozyme properties were combined with robotic principles of controllability and automation to develop nanozyme-based microrobots capable of targeting and treating localized fungal infections at multiple length scales (FIG. 9A). FIG. 9A shows the on-site assembly of individual nanozymes into catalytically active superstructures. The motion dynamics, morphology, and location of catalysis of the structured assemblies can be controlled, creating nanozyme microrobots for targeting fungal infection. FIG. 8B shows the electromagnet core guides the nanozyme microrobot assembly with controllable morphology, position, and motion using programmed algorithms. FIG. 9C shows the programmable dynamic motions via magnetic field modulation enable controlled catalytic activities and targeted treatment.

The disclosed systems can be based on on-site assembly of individual nanozymes into a set of specific reconfigurable superstructures using custom-built electromagnets array with multi-axis motion. An example setup is shown schematically in FIG. 9B. Two coaxially arranged programmable electromagnets are located on either side of a customized container, and an iron core is positioned at the center of electromagnet-1. In-house made iron oxide nanoparticles are building blocks for nanozyme-microrobots. The aqueous iron oxide nanoparticle dispersions are transferred to the container in the working space between the electromagnets. Each of the nanozyme-microrobot configurations is initiated by applying a magnetic field to the dispersion, which is produced by passing direct current through electromagnet-I on the side with the actuatable core. The generated magnetic field from electromagnet-1 induces the magnetization of the iron core and thus the localized magnetic field density near the core within the container, which induces a sea-urchin-shaped nanozyme assemblies next to the iron core. This collection step initializes each of the formations and motions used in these studies.

The shape and motion of the nanozyme microrobots are controlled according to the applied magnetic field from electromagnets, which is harmonized via a programmable microcontroller. According to the ratio of the magnetic field strength generated by the electromagnet-1 and electromagnet-2, the nanozyme assemblies can be variably extended along the y-axis from the core side while oscillating components of the magnetic field enable both structural vibrations as well as enhanced controllability of extension. In addition, the x-z position of the nanozyme assemblies is coupled with the position of the iron core, which can also be controlled by the microcontroller. This controllability allows abundant automated multimodal three-dimensional (3D) motions of nanozyme microrobots.

Although catalytic activities of dispersed iron oxide NP have been well assessed, little is known about the catalysis of structured, mesoscale nanozyme assemblies. Therefore, various magnetic field configurations and corresponding motions, as well as the catalytic properties of nanozyme microrobots, were assessed using the custom-built electromagnets device. 4 different dynamic motions, including rolling, vibrating, gliding, and dabbing (FIG. 9C), which can generate ROS dynamically at different spatial regions and deliver nanozymes through shape transformations, were developed. The rolling motion is induced by translating the initial sea-urchin-like structure across the container surface. For this motion, the position of the electromagnet core is varied, while electromagnet-1 is continuously energized with a constant current. The characteristic rolling motion is caused by a balance between container wall friction, which anchors domains of the structured assemblies near the wall, and the recirculation of nanozyme domains on the outer surface of the assembly. The gliding motion is induced by energizing electromagnet-2 after the initial collection step, which causes the extension of the structured assemblies along the central axis of the electromagnets. In this form, the entire high aspect ratio structure glides along the surface as the electromagnet core is actuated. The rolling and gliding motions can be used for convective fluidic mixing and distribution of generated ROS within the spatial range of actuation.

To achieve the vibration motion, electromagnet-2 is energized with a bias current as well as an oscillating component. This causes the structural ‘spikes’ to rapidly oscillate alternately inward and outward relative to the central axis. Lastly, the dabbing motion is characterized by controllable linear extension along the y-axis. This motion is enabled by applying a simultaneous bias field and oscillating field to electromagnet-2 after an initial x-z plane positioning step. The vibration and dabbing motion can be useful for precisely delivering nanozyme catalysis to specific target locations on distal surfaces.

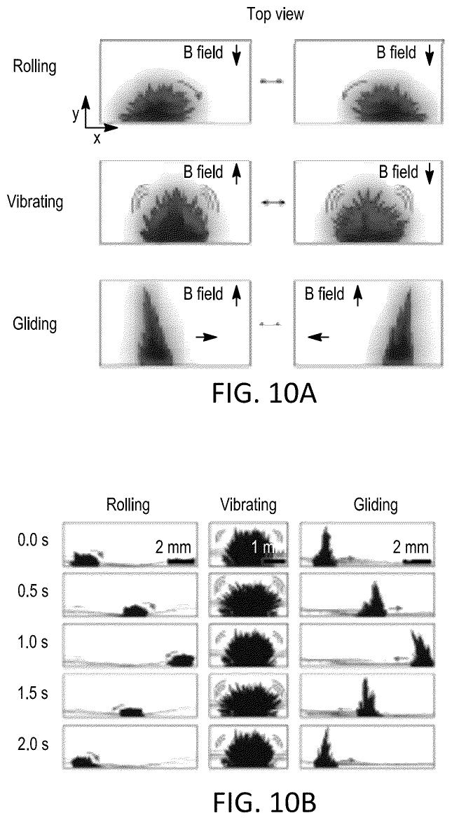

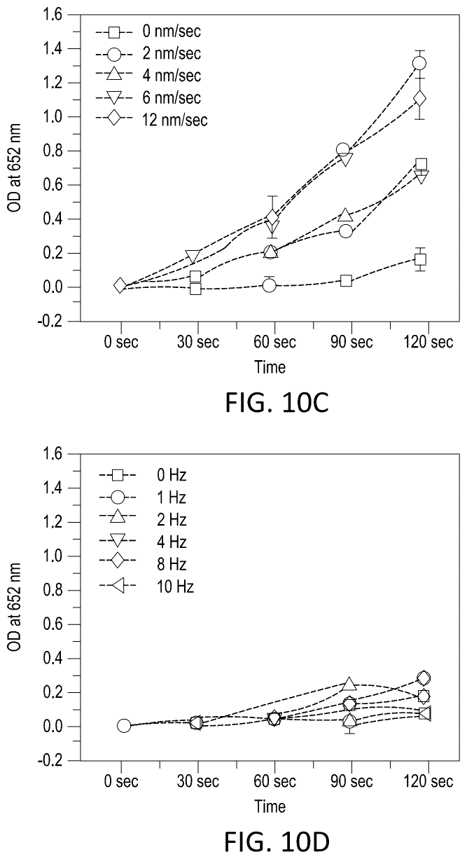

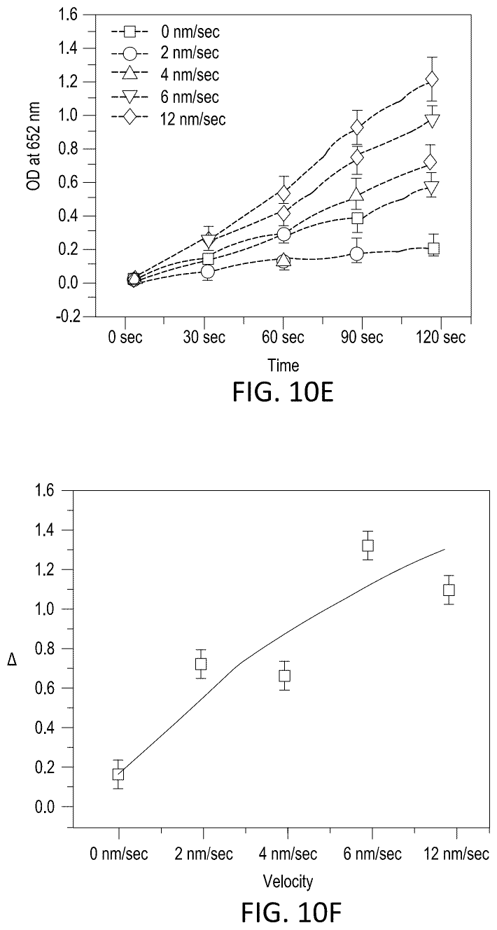

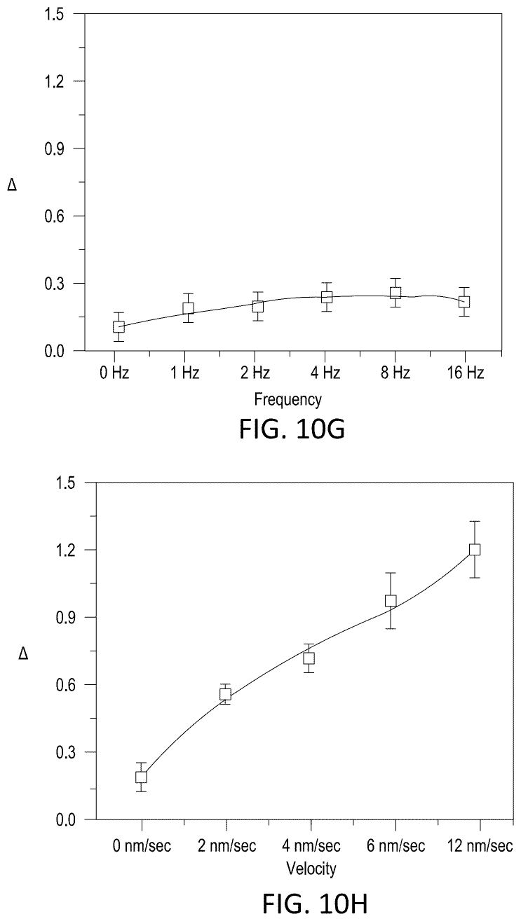

Linear velocities from 0 to 12 mm s⁻¹ with 10 mm path lengths along the x-axis were tested for the rolling and gliding motions, and frequencies from 0 to 16 Hz were considered for the vibrating motion (FIGS. 10A and 10B). Faster linear velocities and higher frequencies lead to more vigorous catalytic reactions for all dynamic motions, whereas minimal catalysis was observed in static conditions (FIGS. 10C-10E). Hence, dynamic motions can activate ROS generation by the nanozyme assemblies, which increase proportionally to linear velocities, leading to significantly enhanced catalytic activity. In the case of rolling and gliding, the highest catalytic activity was about 7 and 6 times greater than that of vibrating nanozyme microrobot, respectively (FIG. 10F-10H).

FIG. 10A shows the catalytic activity in situ generated by motion dynamics. TMB assay demonstrates the generation of ROS on-site from H₂O₂ by the catalytically active (peroxidase-like) nanozyme microrobots. FIG. 10B shows the location of the catalysis controlled by the shape and motion of the nanozyme assemblies. (1 mg mL⁻¹ IONPs). Catalytic activity dynamics of (10C) rolling, (10D) vibrating, and (10E) gliding motions. FIG. 10F shows the velocity dependent catalysis of rolling. FIG. 10G shows the frequency dependent catalysis of vibrating. FIG. 10H shows the velocity dependent catalysis of gliding. The rolling motion shows efficient catalytic activity. Data are mean±standard deviation (n=3).

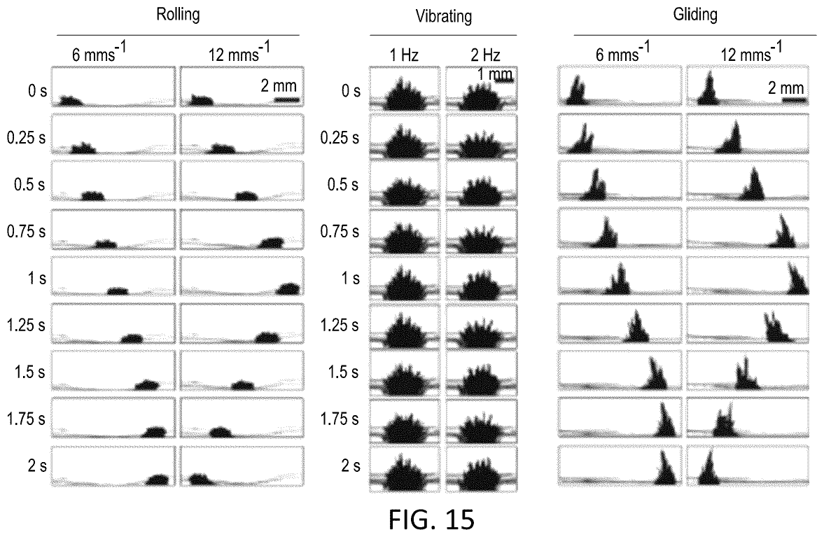

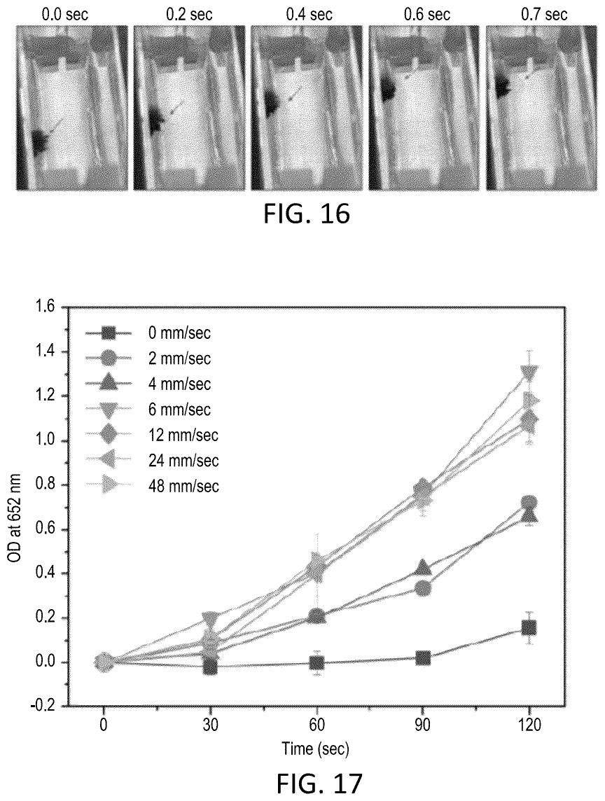

It is noteworthy that increasing vibration frequency results only in incremental enhancement catalytic activity despite creating more pronounced structural movements, indicating that sub-millimeter-scaled amplitude of the vibrating motions can be insufficient to generate sufficient mass transport (FIG. 15). However, centimeter-scale movements of rolling and gliding motions generate fluid convection for mixing and significantly enhance ROS production (FIG. 16). In addition, the shape of the nanozyme microrobot assemblies remains intact during lateral gliding motion. But in a rolling motion, as the assembly circulates, nanoparticles located within the structure are continually exposed to the interface, possibly causing slightly greater catalytic activity. Interestingly, the catalytic activity enhanced by rolling motion saturates above 6 mm s⁻¹ (FIG. 10F and FIG. 17), whereas for gliding, it rises linearly up to 12 mm s⁻¹ and loses structural stability at higher linear velocities (FIG. 10H).

Altogether, nanozyme microrobots with shape-tunable and motion-enhanced catalysis were developed. Giving the limited surface area compared to freely dispersed iron oxide nanoparticles, the nanozyme microrobots display reduced catalytic activity when in a static state, suppressing unnecessarily occurring catalytic reactions. On the other hand, nanozyme microrobots can perform localized catalytic reactions when set in motion providing an effective approach to locally generate and directly deliver ROS to targeted spatial locations through the dynamic motions, thereby avoiding off-target effects.

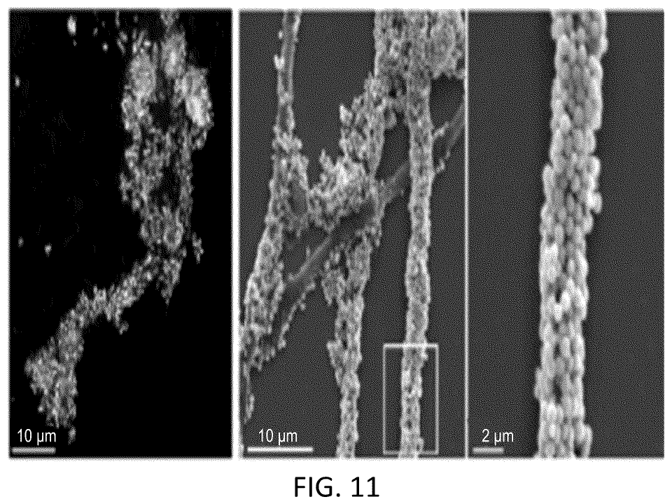

FIG. 11 shows the confocal images, and SEM micrographs demonstrate the binding of nanozymes on C. albicans cells. Data are mean±standard deviation; *P<0.05, **P<0.01, ***P<0.001 by one-way analysis of variance with Tukey's multiple-comparison test (n=12).

Large amounts of nanozymes bound on C. albicans cells were observed when attempting to achieve closer or direct contact with the fungal surface. The nanozymes adhered with remarkably high affinity to both yeast and hyphal forms of C. albicans coating nearly the entire fungal surface as shown using confocal fluorescence and scanning electron microscopy (SEM) (FIG. 11, left and middle). Magnified SEM images show nanozymes forming a coating wrapping around the fungal hyphae (FIG. 11, right). The data indicate the possibility of targeting fungal biofilms.

Precision targeting using experimental models: Next, directed targeting in a palatal mucosal tissue model with localized C. albicans growth was assessed to mimic clinical characteristics of fungal infection on soft tissues. To effectively use the nanozyme-microrobots for precise targeting, an automated and programmable control system was developed. The disclosed system enables control of nanozyme assemblies with three translational degrees-of-freedom. Two of the directions are controlled by servo motors which reposition the electromagnet cores controlling the center of the nanozyme-microrobot, while the third degree-of-freedom is controlled by modulating the currents to precisely and continuously extend the assemblies along the direction of the central axis of the coils.

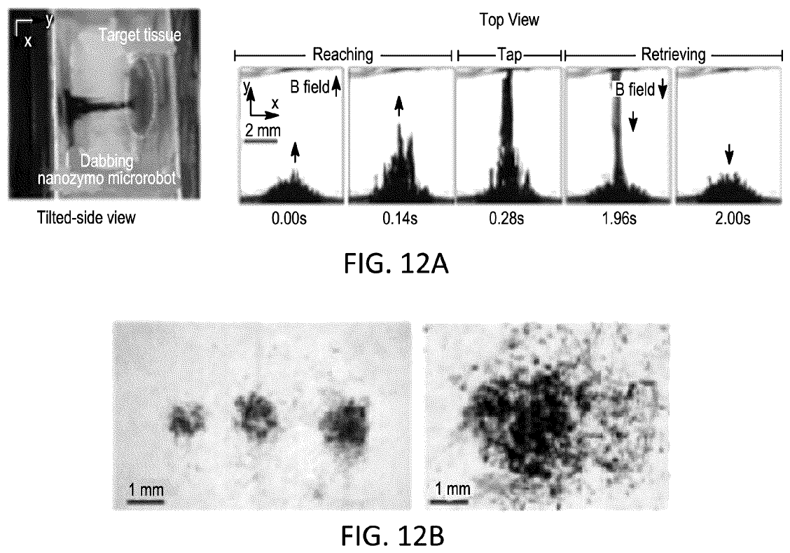

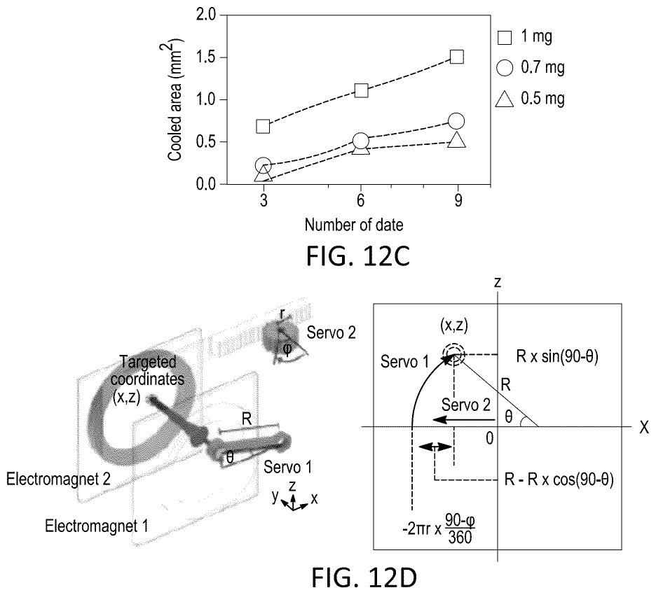

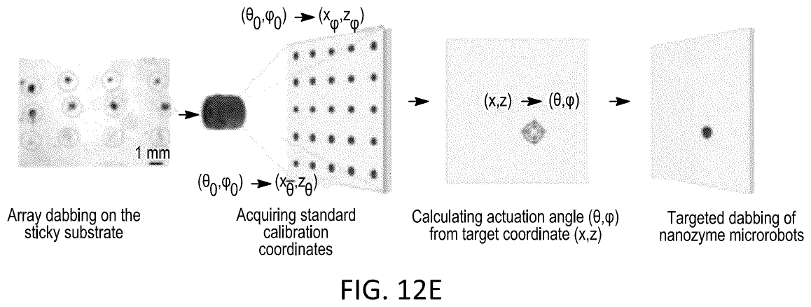

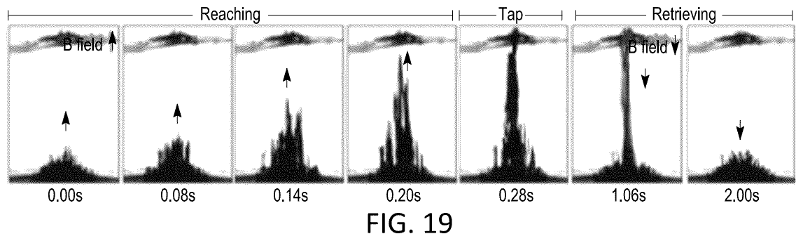

To create the precise dabbing motion, a current was applied to the coil opposite the target to collect nanozymes. While holding the nanozymes against the substrate, the center of the electromagnet core was adjusted in the flat, facial plane of the electromagnetic coil. Once centered in a position aligned with the target in the z-direction, current was applied to the coil behind the target, which caused a linear extension of the structured assembly. However, the assembly typically adjusts shape in abrupt, discrete steps as the nanoparticles reorganize during extension, even when the current is adjusted continuously. To overcome this discrete, step-like extension, a continuously modulating sine wave was applied at 15 Hz superimposed on a bias signal to create a smooth, controllable, and repeatable extension. The high frequency modulated wave aids in the smooth morphological rearrangement of the nanoparticle assemblies during the extension and retraction phases and results in higher aspect ratio superstructures. This results in the dabbing motion used to precisely target tissue surfaces (FIG. 12A and FIG. 19). The time-lapse and the dabbing show remarkable precision and localized nanozyme targeting, whereby the amount and size can be controlled by the number of dabs and concentration of nanozymes (FIGS. 12B-12C). Higher nanozyme concentration and a larger number of dabs show an increase of coated area.

FIG. 12A shows the shape of the dabbing superstructure extending and tapping the targeted surface as visualized under the stereoscope. FIG. 12 B shows the controllable dabbing times and increasing density of nanozyme binding and accurate targeting system. C. albicans coated with nanozymes on the targeted area (Right side image). FIG. 12C shows the area of biofilm coated with nanozymes depends on the IONP concentration. FIG. 12D shows the targeting location is determined by converting servo motor rotation positions to x-z coordinates. FIG. 12E shows a nanozyme dabbing test array that aids in fine-tuning of the positioning accuracy.

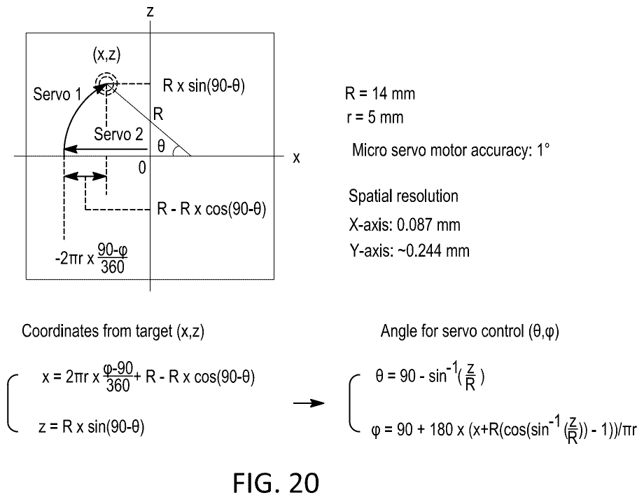

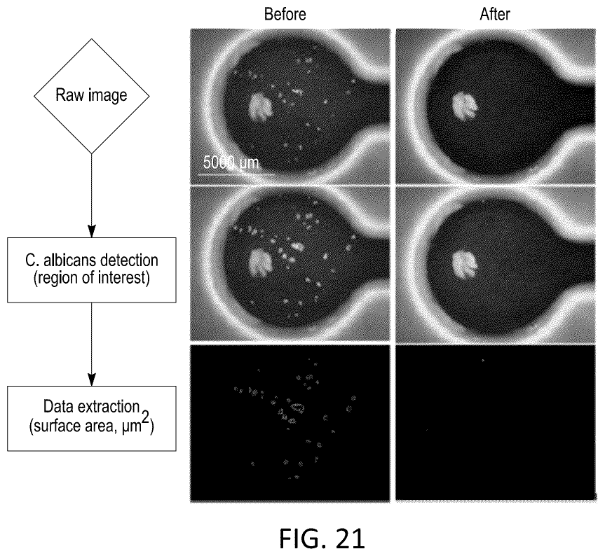

To automate this system, an algorithm was programmed to convert the servo motor positioning angles into the coordinates of the target. The x-axis is controlled by a rack and pinion gear system, while the y-position is determined by a radially sweeping arm (FIG. 12D and FIG. 20). To achieve sub-millimeter precision dabbing motions and further improve precision targeting of nanozymes via the dabbing method that calibrated the nanozyme assemblies to correct for minor positioning errors introduced by sources such as gear positioning and backlash (FIG. 12). Nanozyme assemblies were initially tested on an adhesive substrate during this calibration step. The resulting ‘hitmap’ was used for error correction before nanozyme targeting to tissues.

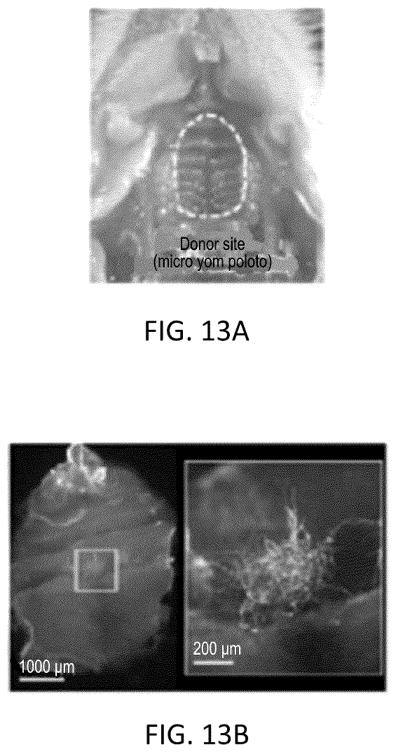

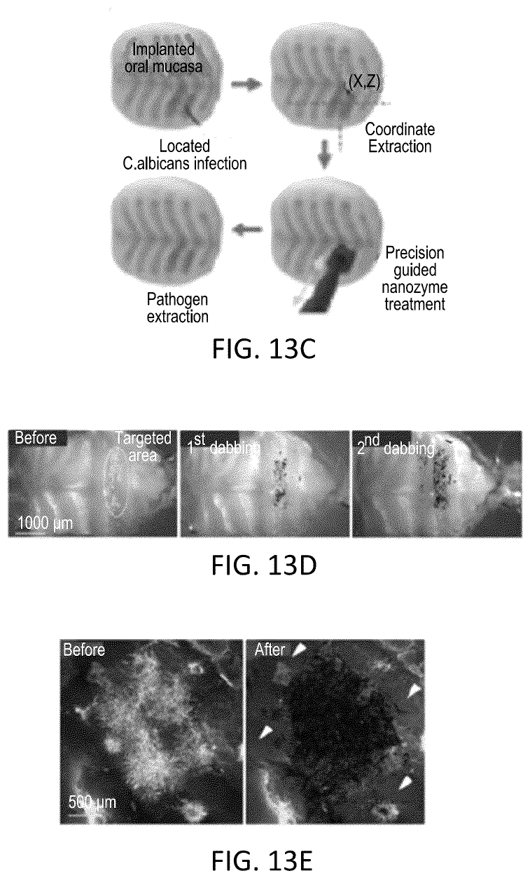

In a clinical setting, Candida infection is characterized by localized white plaques (thrush) on the epithelial tissue comprised mostly of hyphal forms. To test the precision and efficacy of the nanozyme-based robotics approach, an experimental model was used to recapitulate the anatomical features of oral mucosa and localized fungal infection. A primary whole-organ culture was created using explant murine oral mucosa with localized infection sites using C. albicans inoculated on predetermined areas on the palatal tissue (FIG. 13). The data show that C. albicans colonized the mucosal tissue forming a focal infection harboring mostly hyphal cells adhered to the mucosal epithelium (FIG. 13B), similar to those found clinically. The automated targeting was applied via nanozyme-microrobots using dabbing algorithms established in Section 2.4 above towards localized C. albicans infection sites (diagram in FIG. 13C). Spatial coordinates of the targeted sites were extracted using image analysis and used to deliver nanozymes via the guided ‘dabbing approach.’ Imaging analyses showed a controllable dosage of nanozymes on-site to the specimens depending upon the number of dabbing applied (FIG. 13D). Fluorescence images showed that the focal C. albicans infection site was coated by nanozymes, whereas the surrounding epithelial tissue was devoid of nanozymes, suggesting high spatial targeting precision (FIG. 13E).

FIG. 13A shows the explant of murine oral mucosal tissue harvested from the palate. FIG. 13B shows the focal C. albicans infection developed on the oral mucosa characterized by localized hyphal accumulation. FIG. 13C shows a schematic diagram of the coordinate extraction and precision-guided treatment using dabbing nanozyme microrobot. FIG. 13D shows the bright field images showing sequential nanozyme dabbing. FIG. 13E shows the fluorescence imaging showing high spatial precision for targeting the site of fungal infection. Data are mean±standard deviation; **P<0.01, ***P<0.001 by one-way analysis of variance with Tukey's multiple-comparison test (n=8).

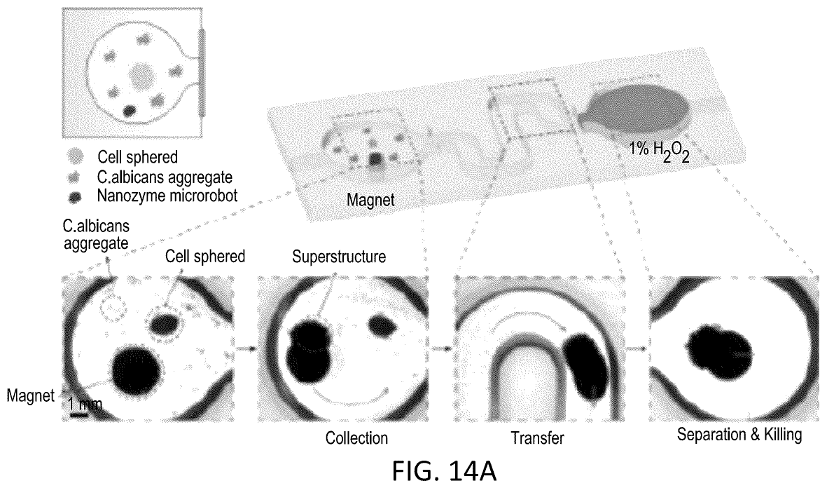

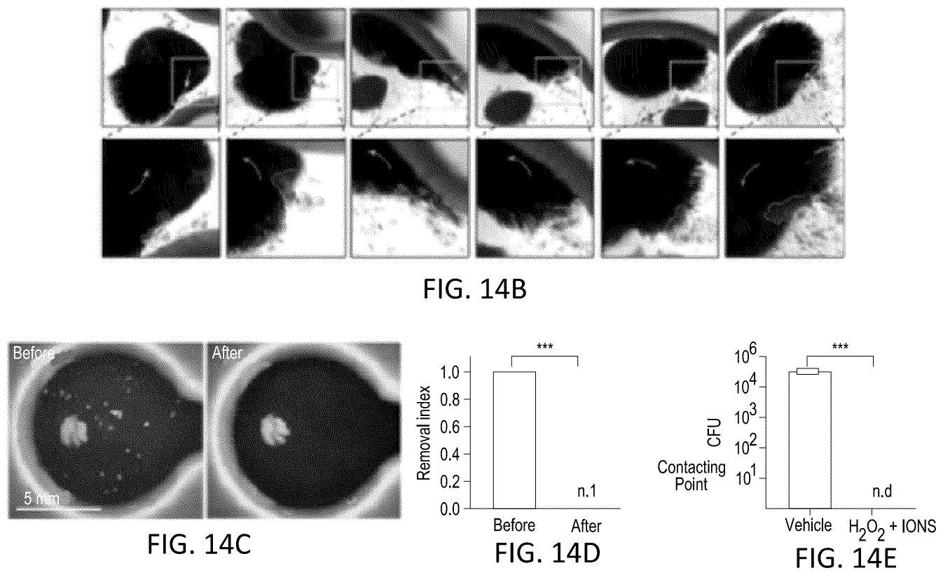

To further assess the antifungal precision and efficacy, a host cell-fungal (human gingival spheroid and C. albicans) model that allows testing fungal cell capturing-transporting to validate nanozyme-binding properties of the microrobots was developed (FIG. 14). In this model, 3D spheroids of human gingival cells were premixed with C. albicans which mimics the oral environment where the host cells and fungal pathogens co-exist (diagram in FIG. 14A). Then, a nanozyme microrobot was magnetically guided to capture and remove the fungal cells without disturbing the human tissue-like spheroid. The captured fungi were transported to a chamber containing hydrogen peroxide.

In the demonstration, the nanozyme assembly could relocate through magnetically controlled motions to capture the fungal aggregates surrounding the spheroid tissue. Time-lapse images show the nanozyme assembly can bind, capture and engulf fungal aggregates while moving across the surface (FIG. 14B). The nanozyme-microrobot minimally binds to the spheroid tissue allowing effective and targeted fungal binding and removal. The effectiveness of fungal capturing was quantitatively assessed by comparing the number of fungal cells before and after the treatment using computational imaging analysis (FIG. 14C and FIG. 21). The data show that nanozyme-robotic approach effectively separated and removed the fungal cells from the mixture (FIG. 14D). Then, the captured C. albicans cells were transferred to a separate chamber containing H₂O₂. After 10 min exposure, the capture fungi were recovered and assessed for viability; the data show complete eradication with no detectable viable cells (FIG. 14E).

FIG. 14A shows a schematic of the experimental platform for testing C. albicans capture in the presence of cell spheroid using nanozyme microrobots. FIG. 14B shows the fungal binding, dragging, and engulfing by the nanozyme microrobot. Close-up images show the fungal aggregates marked by green lines. FIG. 14C provides before and after fluorescence imaging showing that fungal aggregates were effectively removed without binding or disturbing the cell spheroid by a magnetically controlled nanozyme microrobot. FIG. 14D shows the quantitative image analyses showing the complete removal of fungal aggregates. FIG. 14E provides the cell viability counts that show the eradication of the targeted and captured fungal aggregates via catalytic activation of H₂O₂. Data are mean±standard deviation; ***P<0.001 by Student's t-test (n=4).

In summary, microrobotics techniques were employed to control the morphology, on-site catalysis and position of nanozyme assemblies and demonstrate targeting efficacy with microscale precision against C. albicans, a widespread opportunistic fungal pathogen associated with many human infections. Nanozymes were directed to fungal surfaces using planktonic cells, biofilms, aggregates, and tissue infection models. Unexpectedly, the nanozyme assemblies bind avidly to the surface of C. albicans and exploit this natural adhesion property to provide localized targeting modalities. The motion routines and enhanced adhesion are combined to demonstrate unique targeted treatments of C. albicans infections on tissue explants with a customized morphologically adaptive dabbing motion for localized targeting of nanozymes as well as capturing and removal of fungal aggregates from in vivo-like cell spheroids. By applying robotic techniques, a potent mechanism was introduced to enhance fungal targeting and capturing.

Altogether, the results demonstrate that microrobotic techniques for assembly and control of nanozyme constructs provide unique targeting approaches for enhancing the precision and capturing of fungi. The disclosed techniques can be broadly applicable to advancing nanozyme-based robotic assemblies for targeting microbial infections.

### Experimental Section/Methods

Synthesis and Characterization of Iron Oxide Nanoparticles: The iron oxide nanoparticles (IONP) were synthesized by a modified solvothermal method via reducing FeCl₃ at high temperatures. The reaction mixture was prepared by dissolving 975 mg of FeCl3 in 30 mL of ethylene glycol. 1.5 g of sodium acetate was added as a reductant and solvent. Before the heat treatment, the mixture was stirred for 30 min for homogenization. Subsequently, the obtained homogeneous mixture was transferred to a custom-built 50 mL Teflon-lined stainless-steel autoclave and heated at 200° C. for 14 hours. The final products were washed 3 times with Milli-Q water (MilliporeSigma, Burlington, MA, USA) and ethanol. Transmission electron microscopy (TEM) photographs were taken using a FEI Tecnai T12 microscope (FEI, Hillsboro, OR) at an accelerating voltage of 100 kV. The final concentration of IONP was determined by inductively coupled plasma-optical emission spectrometry (ICP-OES) using a Spectro Genesis ICP (SPECTRO Analytical Instruments GmbH, Boschstr, Germany). ICP-OES was calibrated with calibration standards containing known amounts of elements. The diameters of ions are measured using the “imfindcircles” function in MATLAB, which detects circles of a consistent size. The average diameter is found to be 307.8 nm, with a standard deviation of 79.0 nm.

Magnetic Field Control Device: The magnetic fields for the nanozyme microrobots were generated by two electromagnets coaxially assembled at a distance of 8 mm. Each electromagnet was independently energized using a programmable DC power supply (Sorensen XTR60-14, AMETEK Programmable Power, San Diego, USA). An iron core (2 mm in diameter) was mounted on a servo motor to generate the high-gradient magnetic fields at a focused area. The position of the iron core is controlled using a micro servo motor. Between the two electromagnets, a slidable chamber (15×7.3×19 mm³) was designed to accommodate the IONP dispersion and the position of the chamber was controlled by another servo motor. The electromagnets and servo motors were controlled and harmonized by a microcontroller (Arduino Nano, Arduino, Somerville, MA, USA). The Arduino integrated development environment software was used for system coding. To control the motion of nanozyme microrobots, the micro servo motor connected to the iron core was programmed to move between 65° and 115°, which created an up-and-down arc-shaped iron core movement to control the z-coordinate of the nanozyme microrobot. The second micro servo motor (FS90) was used and programmed to control x-axis coordinates with a pinion gear (5 mm radius) attached to the rack of the slidable container for left-to-right motion. A combination of the micro servo motors with the arm and pinion gear allowed high spatial precision (0.087 mm of x-axis, ˜0.244 mm of y-axis) and repeatability in position and timing with linear velocities ranging from 0 to 12 mm s⁻¹. Actuation in all experiments was performed via the application of a magnetic field and servo programmed to achieve the desired motion pattern. The geometries of the slidable container and the body of the device were developed and designed with Onshape, a computer-aided design software (Onshape Inc., Cambridge, USA). The slidable container and the body of the device were fabricated using a low-force stereolithography (SLA) 3D printer (Form 3B, Formlabs Inc., MA, USA) with a biocompatible resin (Surgical Guide). 3D printed parts were rinsed for 20 min in 99.9% isopropanol and then photopolymerized for 30 min under ultraviolet light (405 nm light at 70° C.) (FormCure, Formlabs Inc., MA, USA).

Dynamic motions of Nanozyme Microrobots: four distinct nanozyme microrobot motions were created: rolling, vibrating, gliding and dabbing, named based on the observed dynamics of the nanozyme superstructures. For each motion, nanozymes were first collected from the solution to the wall of the chamber by applying current to the electromagnet-1 for 1.2 s (50 mT, at the center of the chamber). To generate the rolling motion, a side-to-side motion was activated by moving the iron core (on the same side of the electromagnet-1) with the servo motor while maintaining the current at a constant value. The vibrating motion was activated by applying an oscillating field to the electromagnet-2 after the nanozyme collection step. The static field (50 mT, at the center of the container) from the electromagnet-1 was switched off while an oscillating sine wave (varying from 0 to 16 Hz) was generated by electromagnet-2 to produce the vibrating motion. The strength of the magnetic fields was measured to be 12 to 20 mT, at the center of the container. For the gliding motion, the electromagnet-2 was energized by a constant current to reshape the magnetic field such that the nanozyme superstructure was forced to extend along the central axis of the electromagnets. The strength of the magnetic field was measured to be 50 mT at the center of the container. This extended structure was translated from side-to-side with a servo motor. The velocity of the side-to-side motion varied by controlling the angular velocity of the servo motor. To create the dabbing motion, two servo motors were used to reposition the iron core to the targeted position while maintaining a static current. This positioning step was followed by applying a constant bias field with a superimposed sinusoidal field (15 Hz) to the electromagnet-2. This enables the smooth extension of the superstructure. The strength of the magnetic fields was measured to be 10 to 18 mT, at the center of the container. Once the nanozymes were delivered to the targeted tissue or substrate, the electromagnet-2 was de-energized such that the superstructure was fully retracted.

Catalytic Properties of Nanozyme Microrobots: To measure the catalytic properties of nanozyme microrobots, 952.2 μL of sodium acetate (NaAc) buffer (pH 4.5) was added into the actuation chamber followed by 4 μL of TMB (25 mg mL⁻¹ in dimethylformamide) to reach a final concentration of 0.1 mg mL⁻¹. Then, 40 μL of dispersed IONP was added into the chamber at a final concentration of 1 mg mL⁻¹. Immediately before starting the motions (i.e., rolling, vibrating, or gliding), 3.4 μL of H₂O₂ (30%, v/v) was added to the mixture (final concentration: 0.1%). At 30, 60, 90 and 120 seconds, the mixture was transferred to a cuvette, and the absorbance at 652 nm was measured using a spectrophotometer (Beckman Coulter, Inc., Fullerton, CA, USA).

C. albicans Biofilm on Model Surfaces and Non-Surface Attached Fungal Aggregates: Individual 3D-printed acrylic sheets were created as model surfaces with dimensions of 18.0×18.0×0.4 mm³. 3D printing was done using a low-force stereolithography (SLA) 3D printer (Form 3B, Formlabs Inc., MA, USA) with a biocompatible Surgical Guide resin. 3D printed parts were rinsed for 20 min in 99.9% isopropanol and then photopolymerized for 30 min under ultraviolet light (405 nm light at 70° C.) (FormCure, Formlabs Inc., MA, USA). The 3D-printed surfaces were sterilized in an autoclave for 15 min at 121° C., and then coated with filter-sterilized, clarified whole human saliva, which was designed to mimic the denture (abiotic) surface in the oral cavity. C. albicans SN250, a biofilm-forming model fungal pathogen, was grown in ultrafiltered (10-kDa cutoff; Millipore, MA, USA) tryptone-yeast extract (UFTYE, pH 5.5) broth containing 1% (w/v) glucose at 37° C. and 5% CO₂ to mid-exponential phase. The saliva-coated 3D-printed surfaces were inoculated with ˜10⁵ CFU of actively growing C. albicans cells (yeast form) per milliliter in UFTYE (pH 7.0) containing 1% (w/v) sucrose. Biofilms were grown at 37° C. with 5% CO₂ for 12 h. Non-surface attached aggregates of C. albicans were prepared following established protocols. Briefly, planktonic C. albicans cells (10⁵ CFU mL⁻¹, yeast form) were resuspended in filter-sterilized, clarified whole human saliva supplemented with 1% (w/v) sucrose and were incubated at 37° C. for 60 min to form fungal aggregates. C. albicans was stained with concanavalin A (ConA) lectin conjugated with tetramethylrhodamine (555/580 nm; Molecular Probes Inc., Eugene, OR, USA).

Culture of Human Gingival Cells Spheroids and Explant Mucosal Tissue: Human gingival tissue-derived mesenchymal stem cells (GMSCs) were isolated from deidentified gingival tissues obtained during third molar (wisdom tooth) extraction at the University of Pennsylvania under the approved Institutional Review Board (IRB) protocol (IRB #816238). GMSCs were expanded up to passage five in Minimum Essential Medium Alpha (MEM a) media (containing 15% FBS, 2 mM L-glutamine, 100 μM ascorbic acid, 100 U mL⁻¹ penicillin, and 100 μg mL⁻¹ streptomycin). Cells were seeded at a density of 10⁶ mL⁻¹ in ultra-low attachment tubes and grown for 3 days in a humidified tissue culture incubator (37° C., 5% CO₂) to form a 3D spheroid. To model a localized C. albicans infection on the oral mucosal tissue, a primary whole-organ explant culture of murine oral mucosa was used. Briefly, mucosal tissue (4 mm×2 mm) was harvested from the palate of C57BL/6 mice and cultivated in MEM a media (15% FBS, 2 mM L-glutamine, 100 μM ascorbic acid, 100 U mL⁻¹ penicillin, and 100 tg mL⁻¹ streptomycin) at 37° C. with 5% CO₂ for 24 h. The explant cultures were then used to create focal C. albicans infection, as detailed in the section “Focal Candida Infection on Explant Mucosal Tissue Culture.” Precise Fungal Capturing by Nanozyme Microrobots: To test the precise fungal capturing by nanozyme microrobots, a custom-built dual chamber device and an in vivo host-fungal (human gingival cells and C. albicans) model was developed (diagram in FIG. 12A). The 3D-printed device has two chambers (10 mm in diameter, 5-mm deep) connected by a canal (3.5-mm wide, 60-mm long) with a gate on each side. A 3D human gingival spheroid (˜10⁶ cells) and C. albicans aggregates (˜10⁴ CFU) were inoculated and premixed in the first chamber to mimic the oral environment where the host cells and fungal pathogens co-exist. Nanozyme microrobots were magnetically actuated by a neodymium magnet to precisely capture the fungal cells. Then, the captured fungal cells were separated from the mixture by the nanozyme microrobots and transported through the canal to the second chamber (containing 1% H₂O₂), which served as a catalytic “pool” via H₂O₂ activation. After 10 min, the fungal aggregates were collected and serial diluted to determine the number of viable cells (CFU).

Focal Candida Infection on Explant Mucosal Tissue Culture: A primary explant culture of murine oral mucosa was used to create a focal Candida infection that mimics localized infection of the oral cavity by C. albicans. The tissue preparation procedure was conducted under the University of Pennsylvania's Institutional Animal Care and Use Committee (IACUC) protocol (IACUC #806682). To establish a localized C. albicans infection on tissue specimens, hydroxyapatite beads (80-μm particle size, Bio-Rad Laboratories, USA) were used as micro-carriers for fungal cells, which allowed inoculating fungal cells onto the mucosal tissue with microscale precision. Briefly, saliva-coated hydroxyapatite beads (sHA) were pre-incubated with ˜10⁶ CFU mL⁻¹ C. albicans (yeast form) in adsorption buffer (50 mM KCl, 1.0 mM KPO₄, 1.0 mM CaCl₂, 0.1 mM MgCl₂, pH 6.5) for 60 min to allow binding of fungal cells. The beads were washed three times with an adsorption buffer to remove unbound fungal cells. Then, the beads were carefully delivered to a predefined site on the mucosal tissue using a microinjection pipette tip under a microscope (Zeiss Axio Zoom V16). Approximately 20 beads were deposited on each tissue specimen. After the inoculation, tissues were incubated in fresh MSC medium at 37° C. with 5% CO₂ for 12 h to establish a focal Candida infection.

Qualitative and quantitative imaging analysis: Brightfield and fluorescence images were taken with a Zeiss Axio Zoom.V16 upright stereo zoom microscope system (Carl Zeiss Microscopy GmbH, Jena, Germany) with 1× objective (numerical aperture, 0.25). For fluorescence imaging, Samples were stained with Concanavalin A-tetramethylrhodamine conjugate (Molecular Probes) to visualize C. albicans cells. Additionally, high-resolution confocal microscopy was performed using a Zeiss LSM 800 upright confocal microscope (Zeiss, Germany) equipped with a 40× water immersion objective (numerical aperture=1.2) and a 10× objective (numerical aperture=0.7). Computational image processing and quantitative analysis were conducted. Briefly, fluorescence images were imported using ImageJ Fiji software (https://imagej.net/software/fiji/). After denoising, images were segmented based on the fluorescent intensity using the Otsu algorithm, and particle size-based filtering was applied to only include C. albicans aggregates in the images. Regions of interest were created for each C. albicans aggregate, and the total surface area (in μm²) was calculated. The Removal Index (RI) was calculated to evaluate the effectiveness of C. albicans removal by nanozyme microrobots, which is defined as

$$RI = \frac{surface area ( after treatment\_}{surface area before treatment} .$$

Data were presented as not detectable when <2% of fungal cells were detected after the treatment.

Statistical Analysis: Statistical analyses were performed with GraphPad Prism 8.0 (GraphPad Software, CA). Data are represented as mean±standard deviation. Student's t-test was used to compare the mean between two experimental groups. Comparisons of mean between multiple groups were performed using a two-sided one-way analysis of variance (ANOVA) with post hoc Tukey's test. A P value less than 0.05 was considered significant. At least three independent experiments were performed unless otherwise stated.

FIG. 15 provides the time-series images showing the dynamic motions of the nanozyme superstructure. The top-view image sequences show the rolling motion (left, 6 mm s⁻¹ and 12 mm s⁻¹), vibrating motion (middle, 1 Hz and 2 Hz), and gliding motion (right, 6 mm s⁻¹ and 12 mm s⁻¹) with 1.0 mg mL⁻¹ of IONP.

FIG. 16 shows the catalytic activity in situ. TMB assay demonstrates the generation of ROS on-site from H₂O₂ by the catalytically active (peroxidase-like) nanozyme superstructure (1.0 mg mL⁻¹ of IONP).

FIG. 17 shows the dynamics of catalytic activity. Rolling motion, 1.0 mg mL⁻¹ of IONP and 0-48 mm s⁻¹ of linear velocity were tested (n=3).

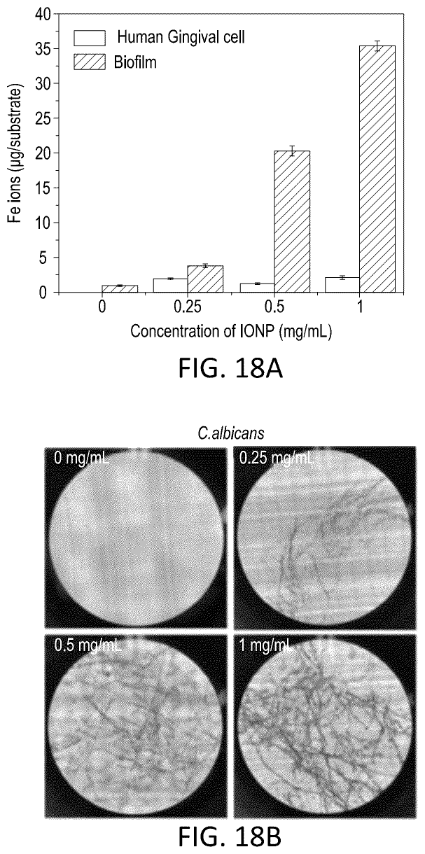

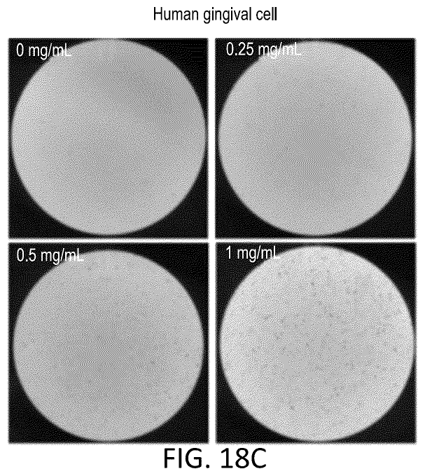

FIG. 18A provides quantitative analysis that shows a trivial amount of IONPs were bound on the human gingival cell layer regardless of the amount of treated IONP (n=3). FIG. 18B shows the optical microscope images of the confluent gingival cell layer after incubation with 0, 0.25, 0.5, and 1 mg/ml of IONP for 10 min. Black dots are bound to IONP on the gingival cell.

FIG. 19 shows time-series images showing the dynamic motions of the dabbing motion.

FIG. 20 shows the coordinate extraction algorithm. One servo motor controls the location of a sliding container through a rack and pinion gear, while the second servo controls the location of the electromagnet core attached to a rotating arm. The servo orientation values were converted to the targeted cartesian coordinates based on this geometry.

FIG. 21 shows the quantification of the fungal capturing and removal by computational image analysis. To quantitatively analyze the amount of fungal cells in the system before and after the treatment, fluorescence images were imported using ImageJ Fiji software and denoised (top). Images were segmented based on the fluorescent intensity using the Otsu algorithm, and particle size-based filtering was applied to only include C. albicans aggregates in the images (middle). Then, regions of interest were created for each C. albicans aggregate, and the total surface area (in μm²) was calculated (bottom).

### Example 4: Nanozyme-Based Robotics Approach for Targeting Fungal Infection

While microrobots have been developed for specific tasks in the medical field, applications in oral and craniofacial health care remain sparse. The few systems that have been introduced are large-scale haptic robots (e.g., YOMI) to help increase predictability and precision during dental implant surgery. However, there are ample opportunities for implementing microrobots and automation to develop new therapeutic approaches. Potential applications include the automated removal of dental biofilms (plaque) on tooth surfaces, orthodontic appliances, and implants. Microrobotic platforms can also be developed to allow precision-guided therapies to promote soft tissue and bone regeneration. By leveraging the cargo-loading ability of microrobots, therapeutic applications can be tailored for drug, stem cell, or growth factor delivery in different oral-craniofacial sites, from deep periodontal pockets and the apical region of the canal to difficult-to-reach temporomandibular spaces to locally stimulate osteogenic or stem cell differentiation. Recent advances in nanotechnology and robotics have enabled the integration of magnetocatalytic nanoparticles with microrobotics principles to target and eradicate biofilms in clinically challenging settings. Here, the catalytic and magnetic properties of iron oxide nanoparticles (NPs) were exploited to introduce a microro-botics platform designed for biofilm treatment and diagnostics (FIG. 7A) using endodontic biofilm models as an exemplar proof-of-concept application.

Targeting Endodontic Biofilms: Incomplete root canal disinfection remains the main cause of treatment failure, leading to persistent endodontic infections and apical periodontitis in conventional antimicrobial and regenerative procedures. The anatomical complexities in the root canal system hinder the effective removal of biofilms, while approaches to diagnose or assess disinfection efficacy are limited. New disinfection modalities such as photon-induced, photo-acoustic streaming; passive ultrasonic irrigation; antimicrobial nanoparticles; and photodynamic therapy have been proposed and/or used for endodontic biofilm treatment. However, these approaches lack controlled targeting of the anatomical complexities and are unable to retrieve biofilm samples for diagnostics. New technologies could enable multi-functionality to access difficult-to-reach surfaces and perform biofilm removal, and microbial detection simultaneously for effective and precise endodontic therapy. Such multi-modal approaches may be achieved using microrobotics.

Magnetically Driven Catalytic Antibiofilm Robots: IONPs have catalytic properties that arise from intrinsic enzyme-like (peroxidase-like) activity that activates H₂O₂ to generate bioactive molecules in situ to disrupt oral biofilms (FIG. 7A). Furthermore, IONPs are widely used in nanomedicine due to their minimal cytotoxicity, excellent physicochemical properties, stability in aqueous solutions, and biocompatibility. One of the first nanoparticle formulations to be approved by the Food and Drug Administration (FDA) for clinical use was an iron oxide nanoparticle contrast agent for magnetic resonance imaging (Feridex, Bayer Healthcare Pharmaceuticals Inc), while another similar preparation (Feraheme, AMAG Pharmaceuticals, Inc) was subsequently approved for the treatment of iron-deficiency anemia. The histopathological analysis of gingival, mucosal, and other tissues, including major organs such as liver and kidney, showed no signs of harmful effects, such as proliferative changes, inflammatory responses, or necrosis, indicating high histocompatibility of both in-house and FDA-approved iron oxide NP formulations with H₂O₂ treatment. Furthermore, NPs can be directed to precise locations using magnetic fields that are generated by permanent magnets or electromagnets. Magnetic actuation allows tether-free controlled motion, enables a wide variety of robotic locomotion strategies, and can readily and harmlessly penetrate biological and synthetic materials and direct robots' motion in confined spaces (FIG. 7B)

The flexibility of “NP building blocks” allows the formation of reconfigurable aggregated microswarms, inclusion in 3-dimensional (3D)-molded soft helicoids, or even direct 3D printing. Here, 2 platforms were developed. The first system uses magnetic forces to concentrate NPs and form structured aggregates (FIG. 7C). These aggregated microswarms are catalytically active robotic structures that are driven by controlled magnetic fields to the apical region of the tooth (FIG. 7D, E). They are magnetically driven to mechanically remove and retrieve the disrupted biofilms. The second platform is designed to be amenable for micromolding with 3D printing techniques to create specific shapes for intracanal locomotion. Miniaturized, helical robots are fabricated from a matrix of biocompatible hydrogel within which NPs are embedded. The robot shape features a double helix having 1.5 turns swept around a central axis. The molds are fabricated using a stereo-lithography 3D printer (FIG. 7F). Navigation of microrobots is controlled by rotating magnetic fields generated by electromagnets to propel through fluids in the desired direction (FIG. 7H). The 3D molded helicoids guided to the apical region can be used to transport bioactives or drugs and release them on-site (i.e., apical region) (FIG. 7G), which may lead to multipurpose applications.

Further development of microrobots can be precisely guided to reach the apical area and used to deliver bioactives or drugs in situ to achieve both chemical disinfection and tissue regeneration. The disclosed subject matter can be used for robotics applications to detect, treat, and remove biofilms associated with other infectious diseases and biofouling of dental/medical devices or implants. The disclosed subject matter can stimulate the utilization of the latest advances in small-scale robotics and nanotechnology that are providing previously unimagined opportunities for new diagnostic and therapeutic approaches with high precision, control, and efficiency.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A diagnostic system comprising:
a small scale robot including a plurality of magnetic nanoparticles (MNPs);
a magnetic control system comprising a magnet, wherein the small scale robot is magnetically aggregated structure of the MNPs under a magnetic field generated by the magnet,
wherein the small-scale robot is configured to disrupt and/or retrieve a target sample from a target area.

2. The diagnostic system of claim 1, wherein the small-scale robot is configured to disrupt and/or retrieve the target sample from the target area through an automated programmable motion and/or positioning.

3. The diagnostic system of claim 1, wherein the small-scale robot is a microscale robot, a miliscale robot, or a centimeter-scale robot.

4. The diagnostic system of claim 1, wherein the target area comprises a square groove, a circular groove, a triangular groove, a crevice, a trench, a space between two or more surfaces, an uneven surface, or combinations thereof.

5. The diagnostic system of claim 1, wherein the small-scale robot is configured to reach and conform to the target area, wherein the target area comprises any exterior surfaces or enclosed surfaces.

6. The diagnostic system of claim 1, wherein the MNPs are configured to be reusable or re-aggregated for an area with different topographies.

7. The diagnostic system of claim 1, wherein the small-scale robot has an extensible and retractable bristle-like configuration, wherein the extensible and retractable bristle-like configuration is configured to have a topography-adaptive property, wherein the topography-adaptive property comprises a stiffness, a shape, length, or a combination thereof.

8. The diagnostic system of claim 1, wherein the magnet comprises a permanent magnet, an electromagnet, or a combination thereof.

9. The diagnostic system of claim 1, wherein the MNPs comprises iron oxide nanoparticles (IONPs), nickel, cobalt, iron, or their alloys or oxides.

10. A diagnostic system comprising:
a small scale robot including a plurality of magnetic nanoparticles (MNPs), wherein the small scale robot is a molded or a 3D printed structure of the MNs in a predetermined shape, wherein the small scale robot is configured to disrupt and/or retrieve a target sample from a target area under a magnetic field.

11. The diagnostic system of claim 8, wherein the predetermined shape comprises a helicoid shape, a vane-like shape, a spherical shape, a bullet-like shape, a spheroid shape, a cylindrical shape, or a spiral-like shape

12. The diagnostic system of claim 10, wherein the small-scale robot is configured to rotate and propel under the magnetic field.

13. The diagnostic system of claim 9, wherein the small scale robot is a microscale robot, a miliscale robot, or a centimeter-scale robot.

14. The diagnostic system of claim 9, wherein the target area comprises a square groove, a circular groove, a triangular groove, a crevice, a trench, a space between two or more surfaces, an uneven surface, or combinations thereof.

15. The diagnostic system of claim 9, wherein the MNPs are configured to be reusable or re-aggregated for an area with different topographies.

16. The diagnostic system of claim 9, wherein the MNPs comprises iron oxide nanoparticles (IONPs), nickel, cobalt, iron, or their alloys and oxides.

17. A method for retrieving a target sample, comprising:
applying a magnetic field to magnetic nanoparticles (MNPs);
forming a small scale robot by aggregating the MNPs into a predetermined shape;
introducing the small scale robot into a target area; and
disrupting and/or collecting the target sample from the target area by applying a controlled magnetic field that actuates automated programmable motion and positioning of the small-scale robot.

18. The method of claim 15, further comprising
adjusting mechanical properties of the small-scale robot based on the target area, wherein the mechanical properties comprise a shape, a length, a stiffness, or combinations thereof.

19. The method of claim 15, further comprising
analyzing the collected samples for identifying a pathogen, by-products of the pathogen, a composition of the target area, or a combination thereof, wherein the pathogen includes a bacteria, a fungus, a virus, archae, protozoa, algae, and the by-products can include biomolecules and metabolites, or combinations thereof.

20. The method of claim 15, further comprising
reshaping the small-scale robot to reach an area with different topographies.

21. The method of claim 15, wherein the small scale robot is a microscale robot, a miliscale robot, or a centimeter-scale robot.

22. The method of claim 15, wherein the target area comprises a square groove, a circular groove, a triangular groove, a crevice, a trench, a space between two or more surfaces, an uneven surface, or combinations thereof.

23. The method of claim 15, wherein the small scale robot has an extensible and retractable bristle-like configuration.

24. The method of claim 15, wherein the small-scale robot is configured to reach and conform to the target area, wherein the target area comprises any exterior surfaces or enclosed surfaces.

25. The method of claim 15, wherein the MNPs comprises iron oxide nanoparticles (IONPs), nickel, cobalt, iron, or their alloys and oxides.

26. The method of claim 15, the method further comprising identifying the target sample by detecting components of the sample through an analyzer.
